(19) United States
(12) Patent Application Publication
(10) Pub. No.: US 2010/0179092 A1
(43) Pub. Date: Jul. 15, 2010

(54) HUMAN COMPLEMENT C3 DERIVATIVES WITH COBRA VENOM FACTOR-LIKE FUNCTION

(76) Inventors: David C. Fritzinger, Honolulu, HI (US); Carl-Wilhelm Vogel, Honolulu, HI (US)

Correspondence Address: MORRISON & FOERSTER LLP, 755 PAGE MILL RD, PALO ALTO, CA, 94304-1018, US

(21) Appl. No.: 12/513,665

(22) PCT Filed: Nov. 15, 2007
(86) PCT No.: PCT/US07/24163

§ 371 Date: Mar. 9, 2010

Related U.S. Application Data
(60) Provisional application No. 60/859,330, filed on Nov. 15, 2006.

(51) Int. Cl. A61K 38/16; C07K 14/00; C07H 21/04; A61P 43/00; C12N 15/63; C12P 21/02; C12N 5/10; C12N 1/21; C12N 1/19

(52) U.S. Cl. 514/12; 530/350; 536/23.5; 435/320.1; 435/69.1; 435/367; 435/358; 435/252.33; 435/252.31; 435/254.2; 435/254.21; 435/254.23; 435/348

(57) ABSTRACT

The invention provides modified human complement C3 proteins, comprising a human C3 protein, wherein amino acid residues in the human C3 protein are substituted with a corresponding portion of a Cobra Venom Factor (CVF) protein, and wherein one or more amino acid residues in the CVF portion of the modified human complement C3 protein are further modified.

### RELATED APPLICATIONS

This application claims priority benefit to the provisional application 60/859,330, filed on Nov. 15, 2006, the contents of which are incorporated by reference herein in its entirety.

### STATEMENT REGARDING FEDERALLY SPONSORED RESEARCH OR DEVELOPMENT

Not applicable

### FIELD OF THE INVENTION

The invention relates generally to modified Human Complement C3 having a substitution of a portion of a human C3 protein with a corresponding portion of a Cobra Venom Factor (CVF) protein.

### BACKGROUND OF THE INVENTION

The third component of complement, C3, plays a pivotal role in both the classical and alternative pathways of complement activation, and many of the physiological C3 activation products have important functions in the immune response and host defense (for review see Müller-Eberhard, H. J. (1988) “Molecular Organization and Function of the Complement System,” Ann. Rev. Biochem., 57:321-347). Human C3 is a two-chain glycoprotein with a molecular weight of approximately 185,000 Daltons. It is synthesized as single-chain pre-pro-C3 which undergoes subsequent processing by removal of four arginine residues between the α- and β-chains. The primary sequence of human C3 is known from molecular cloning. Full or partial sequence information of C3 from other mammalian species as well as non-mammalian species is available, including mouse, rat, guinea pig, chicken, cobra, Xenopus, and lamprey. The human C3 gene is 42 kb in length and includes 41 exons, ranging in size from 52 to 213 bp. C3 is a highly unusual multi-functional protein. The human C3, including its various activation products, specifically interacts with approximately twenty different plasma proteins or cell surface receptors. For some ligands of C3, including Factor H, properdin, Factor B, and the complement receptors CR1, CR2, CR3, and C3a receptor binding sites have been proposed or assigned to more or less defined regions of the C3 polypeptide.

The major activation product of C3 is C3b. C3b plays a central role in the formation of the alternative pathway C3/C5 convertase. In the alternative pathway, the activated form of C3, C3b, is a structural subunit of the C3 convertase. This bimolecular enzyme consists of C3b and Bb, the activated form of complement. The formation of this enzyme requires initial binding of C3b to Factor B. The weak complex C3b,B is subsequently cleaved by factor D in the presence of Mg²⁺, resulting in the enzymatically active C3/C5 convertase C3b,Bb and in release of the activation peptide Ba. The C3/C5 convertase cleaves C3 and C5 by hydrolyzing a single peptide bond in the α-chains of the two substrates. The C3 convertase activates C3 by cleaving the molecule into C3b and the anaphylatoxin, C3a. The C3b molecule will bind to a cell or particle in close proximity to the C3 convertase. Eventually, the bound C3b will allow for the activation of C5 into C5b and the anaphylatoxin, C5a. C5 activation occurs by the same C3b,Bb enzyme that can cleave C5 when it is bound to an additional C3b molecule to produce a trimolecular complex composed of (C3b)₂,Bb. This C5-cleaving trimolecular enzyme is called C5 convertase. Inasmuch as the activation of both C3 and C5 occurs at the identical active site in the Bb subunit, the enzyme is also called C3/C5 convertase; and only one EC number has been assigned (EC 3.4.21.47).

An unusual structural property of C3 is the presence of an intramolecular thioester in the α-chain. Upon activation of C3 to C3b, the thioester becomes highly reactive and is responsible for the covalent attachment of C3b to cellular and other particular targets. The structural change which accompanies cleavage of the thioester allows the subsequent binding of factor B and its activation. The C3b,Bb enzyme is very labile, exhibiting spontaneous decay-dissociation into the two subunits C3b and Bb with an intrinsic half life of 1.5 minutes at 37° C. The C3b,Bb enzyme is stabilized by properdin. The thioester in C3 undergoes slow spontaneous hydrolysis, resulting in the formation of a form of C3 called iC3 or C3(H₂O). iC3 assumes C3b-like functions and can form a fluid-phase convertase with factors B and D in serum. Spontaneous hydrolysis of the thioester and the ensuing low grade activation of C3 by the iC3,Bb convertase is believed to be responsible for the initial deposition of C3b on target cells or particles, leading to activation of the alternative pathway on so-called activator surfaces.

In addition to the fast spontaneous decay-dissociation, the C3b,Bb enzyme is subject to stringent control. The enzyme is disassembled by factor H, and C3b is inactivated by the combined action of factors H and I. In the presence of factor H, factor I cleaves the α′-chain of C3b at two cleavage sites. The resulting C3b derivative, called iC3b, can no longer form a convertase with factor B. Factor I can cleave the α′-chain at a third site, which causes the generation of the two C3 fragments C3c and C3dg. For the third cleavage by factor I, the C3b receptor, CR1 serves as co-factor.

Cobra venom contains a structural and functional analog of C3 called cobra venom factor (CVF). CVF is a three-chain glycoprotein with a molecular mass of approximately 150,000 Daltons. This molecule can bind factor B in human and mammalian serum to form the complex, CVF,B, which is also cleaved by factor D in the presence of Mg²⁺ into the bimolecular enzyme CVF,Bb and Ba. The bimolecular complex CVF,Bb is a C3/C5 convertase that activates C3 and C5 analogously to the C3/C5 convertase formed with C3b. CVF and mammalian C3 have been shown to exhibit several structural similarities including immunological cross-reactivity, amino acid composition, circular dichroism spectra and secondary structure, and electron microscopic ultrastructure. Initial N-terminal amino acid sequence comparisons have demonstrated sequence homology with C3 and have led to the suggestion that CVF structurally resembles C3c. The structural homology between CVF and C3 and the chain relationships were confirmed by the molecular cloning of CVF, which revealed an overall similarity at the protein level of approximately 70 percent to mammalian C3s and over 90 percent when compared to cobra C3.

Despite these functional and structural similarities between CVF and C3, the two molecules exhibit important structural differences. For example, whereas C3 is a two-chain molecule with an apparent molecular mass, dependent on the species, of 170 to 190 kDa, CVF is a three-chain molecule with an apparent molecular mass of 149 kDa that resembles C3c, one of the physiologic activation products of C3. Another significant structural difference between C3 and CVF lies in their glycosylation: CVF has a 7.4% (w/w) carbohydrate content consisting mainly of N-linked complex-type chains with unusual α-galactosyl residues at the non-reducing termini. In contrast, human and rat C3 exhibit a lower extent of glycosylation with different structures of their oligosaccharide chains.

The CVF and human C3 molecules and the resulting convertases also exhibit important functional differences. For example, the C3b,Bb enzyme generated during complement activation is surface bound. In contrast, the CVF,Bb enzyme is a fluid-phase enzyme (like iC3,Bb). Another functional difference between C3b,Bb and CVF,Bb lies in the C5 convertase activities. In order for C5 to be cleaved by a C5 convertase, it needs to be bound to either C3b or CVF. However, for C5 cleavage to occur by the C3b,Bb enzyme, C5 has to be bound to a different C3b molecule than the one that is part of the C3b,Bb enzyme. In contrast, C5 is bound by the same CVF molecule that carries the Bb catalytic subunit. This property of the CVF,Bb enzyme to bind C5 is probably the reason for its ability to exhibit fluid phase C5 convertase activity, whereas the C5 convertase activity of the C3b,Bb enzyme is confined to the surface of a particle. In addition, both enzymes have been shown to differ somewhat in their kinetics of C3 hydrolysis. Based on the kcat/Km, the catalytic efficiency is approximately eight-fold greater for C3b,Bb compared to CVF,Bb.

In terms of functional consequences, the two most significant differences between CVF,Bb and C3b,Bb are the intrinsic stability of the CVF,Bb enzyme (the C3b,Bb enzyme has an intrinsic half-life of 1.5 minutes at 37° C., whereas the CVF,Bb enzyme has an intrinsic half-life of approximately seven hours) and CVF,Bb resistance to the regulatory proteins factors H and I. Once the CVF,Bb enzyme has formed, it will continue to activate C3 and C5, leading to complement consumption. The combination of the long intrinsic half-life and the resistance to regulation of the CVF-containing enzymes allows CVF to continuously activate C3 and C5 (and subsequently other complement components), ultimately resulting in depletion of the serum complement activity. Ever since it was demonstrated over 30 years ago that CVF can be administered safely to laboratory animals in order to deplete their plasma complement, CVF has become an important investigational tool to study the various biological functions of complement in immune response, host defense, and pathogenesis of disease by comparing normal (complement-sufficient) animals with CVF-treated (complement-depleted) animals.

Based on the involvement of the complement system in multiple diseases, including diseases of major prevalence, the last decade has seen the development of multiple anti-complementary agents to interfere with the unwanted complement activation process in these disease states. All complement-oriented drug development attempts are based on inhibiting the activation of complement, while CVF acts by depleting complement in serum. CVF is a complement inhibitor that acts through a mechanism of exhaustive activation which subsequently leads to depletion. As a matter of fact, CVF is frequently used as the standard to evaluate the anti-complement activity of other drugs. Whereas CVF exhibits this powerful anti-complement activity, it is not suitable for human application because of its immunogenicity.

Of interest for the treatment of diseases of complement activation is a C3-type molecule which combines the non- or low immunogenicity of C3, with the complement-depleting function of CVF. For example, Vogel and Fritzinger in PCT/US2005/05119 (WO 05/107785) disclosed modified human complement C3 proteins comprising substitutions of human C3 protein, with various corresponding portions of a cobra venom factor protein (CVF) which result in human C3 proteins with CVF functions, but with substantially reduced immunogenicity. Additionally, Kolln et al. in U.S. patent application Ser. No. 10/884,813 (U.S. Patent Publication No. US 2005-0079585 A1) provides human C3 derivatives having complement-modulating activity. These human C-3 derivatives contain complement C3 with the carboxy-terminal part being replaced by a corresponding part of CVF.

Because of the potential to treat diseases of complement activation with a C3-type molecule which combines the non- or low immunogenicity of C3 and the complement-depleting function of CVF, it is desirable to take advantage of the extensive structural similarity between CVF and C3 and to further generate modified human C3 with the desired complement-depleting function of CVF and lower immunogenicity than CVF.

### SUMMARY OF THE INVENTION

The invention provides a modified human complement C3 protein, comprising a human C3 protein, wherein the amino acid residues in the human C3 protein corresponding to amino acid residues about 1496 to about 1663 of SEQ ID NO:1 (prepro-human C3) are substituted with a corresponding portion of a Cobra Venom Factor (CVF) protein, and wherein one or more amino acid residues in the CVF portion of the modified human complement C3 protein are further modified.

The invention also provides a modified human C3 protein, comprising a human C3 protein, wherein amino acid residues in the human C3 protein corresponding to amino acid residues about 1496 to about 1663 of SEQ ID NO:1 are substituted with a corresponding portion of a Cobra Venom Factor (CVF) protein, wherein the CVF portion of the modified human C3 protein comprises a further substitution of one or more amino acid residues in one or more regions selected from the group consisting of amino acid residues about 1499 to about 1501, amino acid residues about 1507 to about 1510, amino acid residues about 1519 to about 1550, and amino acid residues about 1596 to about 1617. In some embodiments, the one or more regions are selected from the group consisting of amino acid residues about 1499 to about 1501, amino acid residues about 1507 to about 1510, amino acid residues about 1519 to about 1550, amino acid residues about 1519 to about 1524, amino acid residues about 1528 to about 1532, amino acid residues about 1596 to about 1617, amino acid residues about 1596 to about 1611, amino acid residues about 1598 to about 1608, amino acid residues about 1598 to about 1600, and amino acid residues about 1607 to about 1608. In some embodiments, the corresponding portion of the CVF protein is amino acid residues 1475 to 1642 of SEQ ID NO:2.

In some embodiments, amino acids in the regions such as amino acid residues about 1499 to about 1501, amino acid residues about 1507 to about 1510, amino acid residues about 1519 to about 1550, amino acid residues about 1519 to about 1524, amino acid residues about 1528 to about 1532, amino acid residues about 1596 to about 1617, amino acid residues about 1596 to about 1611, amino acid residues about 1598 to about 1608, amino acid residues about 1598 to about 1600, and amino acid residues about 1607 to about 1608 in the modified human C3 protein are substituted with a different amino acid, for example, one or more corresponding amino acids of SEQ ID NO:1 or a conservative substitution thereof.

Examples of the substitutions of amino acid residues in the CVF portion of the modified human complement C3 protein include: 1) T1499D and L1501K; 2) I1507R, G1508D, N1509E, and V1510L; 3) S1519F, S1520I, L1521Q, N1522K, H1523S, and Q1524D; 4) D1528T, V1529L, P1530E, L1531E, and Q1532R; 5) 1519-1550 replaced with corresponding amino acid residues of SEQ ID NO:1; 6) 1596-1617 replaced with corresponding amino acid residues of SEQ ID NO:1; 7) 1596-1611 replaced with corresponding amino acid residues of SEQ ID NO:1; 8) V1598L, N1599D, D1600N, S1607L, and R1608S; 9) V1598L, N1599D, and D1600N; or 10) S1607L and R1608S.

In some embodiments, the further substitution of one or more amino acid residues is in the region of amino acid residues about 1499 to about 1501. In some embodiments, the further substitution of one or more amino acid residues is a substitution with one or more corresponding amino acids of SEQ ID NO:1 or conservative substitutions thereof. In some embodiments, the further substitution of one or more amino acid residues is T1499X and L1501Z, wherein X is amino acid residue D or a conservative substitution thereof and Z is amino acid residue K or a conservative substitution thereof. In some embodiments, the further substitution of one or more amino acid residues is T1499D and L1501K.

In some embodiments, the further substitution of one or more amino acid residues is in the region of amino acid residues about 1519 to about 1550. In some embodiments, the further substitution of one or more amino acid residues is a substitution with one or more corresponding amino acids of SEQ ID NO:1 or conservative substitutions thereof. In some embodiments, the further substitution of one or more amino acid residues is a substitution with corresponding amino acids 1519-1550 of SEQ ID NO:1 or conservative substitutions thereof.

In some embodiments, the further substitution of one or more amino acid residues is in the region of amino acid residues about 1598 to about 1600. In some embodiments, the further substitution of one or more amino acid residues is V1598X, N1599Z1, and D1600Z2, wherein X is amino acid residue L or a conservative substitution thereof, Z1 is amino acid residue D or a conservative substitution thereof, and Z2 is amino acid residue N or a conservative substitution thereof. In some embodiments, the further substitution of one or more amino acid residues is V1598L, N1599D, and D1600N.

In some embodiments, the further substitution of one or more amino acid residues is in the regions of amino acid residues about 1499 to about 1501 and amino acid residues about 1519 to about 1550.

In some embodiments, the human C3 protein is a single chain protein. In some embodiments, the modified human C3 protein is cleaved into at least two chains in a form that resembles native human C3. In some embodiments, the modified human C3 protein is proteolyticly cleaved to release a portion therefrom (e.g., a portion like a C3a).

In some embodiments, the modified human C3 protein is a mature protein. In some embodiments, the modified human C3 protein has 1 to about 19 amino acids at the N-terminus that are not part of human C3 or CVF. In some embodiments, the modified human C3 protein contains the signal sequence. In some embodiments, the signal sequence is a non-human C3 signal peptide, such as a Drosophila signal sequence.

The invention also provides compositions comprising one or more modified human C3 proteins described herein. In some embodiments, the compositions further comprise a pharmaceutically acceptable carrier.

The invention also provides polynucleotides comprising a nucleic acid sequence encoding any of the modified human C3 protein described herein. The invention also provides vectors and host cells comprising the polynucleotides.

The invention also provides methods for producing the modified human C3 protein comprising culturing a host cell comprising a polynucleotide comprising a nucleic acid sequence encoding the modified human C3 protein under a condition that the modified human C3 protein is expressed; and purifying the expressed modified human C3 protein.

The invention also provides methods for depleting complement in an individual comprising administering to the individual a modified human C3 protein in an amount effective for the depletion of complement. Such methods can be used for treating and preventing conditions or diseases associated with undesirable complement activation, such as, autoimmune diseases and reperfusion injuries. In some embodiments, the disease is selected from the group consisting of asthma, systemic lupus erythematosus, glomerulonephritis, rheumatoid arthritis, Alzheimer's disease, multiple sclerosis, myocardial ischemia, reperfusion, sepsis, hyperacute rejection, transplant rejection, cardiopulmonary bypass, myocardial infarction, angioplasty, nephritis, dermatomyositis, pemphigoid, spinal cord injury and Parkinson's disease. The administration can be local (e.g., into an organ, subcutaneously, into a cavity, or into a tissue) or systemic (e.g., intravenously or intraperitoneally).

The invention also provides the use of the modified human C3 protein for the depletion of complement. In some embodiments, the use is for treating and preventing a disease selected from the group consisting of asthma, systemic lupus erythematosus, glomerulonephritis, rheumatoid arthritis, Alzheimer's disease, multiple sclerosis, myocardial ischemia, reperfusion, sepsis, hyperacute rejection, transplant rejection, cardiopulmonary bypass, myocardial infarction, angioplasty, nephritis, dermatomyositis, pemphigoid, spinal cord injury and Parkinson's disease.

The invention also provides methods for depleting complement by an ex vivo treatment, comprising transfusing circulation of an individual through a matrix bearing the modified human C3 protein to remove complement. The methods may further comprises a step of removing (e.g., by dialysis) anaphylactic peptides (C3a and C5a) and other low molecular weight inflammatory mediators (e.g. histamine and nitric oxide) prior to the decomplemented blood (or plasma) being returned to the individual.

### BRIEF DESCRIPTION OF THE DRAWINGS

FIG. 1 depicts the chain structures of human C3 and CVF with shaded portions present in the mature proteins.

FIGS. 2A-2D provide an alignment between prepro-CVF (SEQ ID NO:2) from Naja naja cobra and human prepro-C3 (SEQ ID NO:1) amino acid sequences showing the corresponding regions of the two proteins.

FIGS. 3A-3H show nucleic acid and amino acid sequence of human prepro-C3. The NH2- and C-termini of the α- and β-chains, functionally important regions, and known ligand binding sites are indicated. Amino acid residue numbering starts at the NH2-terminus of the pre-pro-C3 molecule.

FIG. 4 shows the percentage of Factor B cleavage over time by the modified human C3 proteins including HC3-1496, HC3-1496-2, HC3-1496-3, HC3-1496/1617, HC3-1496-8, and HC3-1496-9 or control (native CVF or Human C3b).

FIG. 5 shows the percentage of C3 converted by the cleavage of the C3a peptide over time by the modified C3 proteins including HC3-1496, HC3-1496-2, HC3-1496-3, HC3-1496/1617, HC3-1496-8, and HC3-1496-9 or control (native CVF).

FIG. 6 shows the percentage of in vitro complement depleted with increasing concentrations of modified C3 proteins including HC3-1496, HC3-1496-2, HC3-1496-3, HC3-1496/1617, HC3-1496-8, and HC3-1496-9 or control protein (native CVF).

FIG. 7 shows the percentage of in vitro complement depleted with increasing concentrations of modified C3 protein (HC3-1496-13) or control protein (HC3-1496).

FIG. 8 shows the relative amount of complement activity in vivo over time after injection of the modified C3 protein (HC3-1496) or control (native CVF).

### DETAILED DESCRIPTION OF THE INVENTION

The invention provides modified human complement C3 proteins and compositions thereof, and methods of using the compositions for complement depletion.

### DEFINITIONS

It should be noted that, as used herein, the singular form “a”, “an”, and “the” includes plural references unless indicated otherwise.

As used herein, the term “native” is meant to naturally occurring, i.e., is obtainable in nature.

As evident to one skilled in the art, “modified human C3 protein” means the sequence is altered compared to a native human C3 protein.

The terms “polypeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides of this invention can occur as single chains or associated chains.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, α-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S(“thioate”), P(S)S (“dithioate”), (O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

As used herein, “vector” means a construct, which is capable of delivering, and preferably expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

As used herein, the term “treatment” is an approach for obtaining beneficial or desired clinical results. Desirable effects of treatment include, but are not limited to, improvement in any aspect of a disease or a condition and alleviation of one or more symptoms associated with the disease or the condition.

As used herein, the term “prevention” includes providing prophylaxis with respect to occurrence or recurrence of a disease or a condition in an individual. An individual may be predisposed to the disease or the condition but has not yet been diagnosed with the disease or the condition.

As used herein, an “effective dosage” or “effective amount” of drug, compound, or pharmaceutical composition is an amount sufficient to effect beneficial or desired results. For prophylactic use, beneficial or desired results include results such as eliminating or reducing the risk, lessening the severity, or delaying the outset of the disease, including biochemical, histological and/or behavioral symptoms of the disease, its complications and intermediate pathological phenotypes presenting during development of the disease. For therapeutic use, beneficial or desired results include decreasing one or more symptoms resulting from the disease (biochemical, histological and/or behavioral), including its complications and intermediate pathological phenotypes presenting during development of the disease, increasing the quality of life of those suffering from the disease, decreasing the dose of other medications required to treat the disease, enhancing effect of another medication, and/or delaying the progression of the disease of patients. An effective dosage can be administered in one or more administrations. For purposes of this invention, an effective dosage of drug, compound, or pharmaceutical composition is an amount sufficient to accomplish prophylactic or therapeutic treatment either directly or indirectly. As is understood in the clinical context, an effective dosage of a drug, compound, or pharmaceutical composition may or may not be achieved in conjunction with another drug, compound, or pharmaceutical composition. Thus, an “effective dosage” may be considered in the context of administering one or more therapeutic agents, and a single agent may be considered to be given in an effective amount if, in conjunction with one or more other agents, a desirable result may be or is achieved.

An “individual” or a “subject” is a mammal, more preferably a human. Mammals also include, but are not limited to, farm animals, sport animals, pets, primates, horses, dogs, cats, mice and rats.

Reference to “about” a value, a parameter or an amino acid position herein includes (and describes) embodiments that are directed to that value, parameter, or amino acid position per se.

When “about” refers to an amino acid residue in a modified human C3 protein (from CVF substitution), the term “about” refers to a range of plus and/or minus 35 amino acids or less for the position. In some embodiments, the term “about” refers to a range of plus and/or minus 30 amino acids or less, 25 amino acids or less, 20 amino acids or less, 15 amino acids or less, 10 amino acids or less, 5 amino acids or less, or 2 amino acids or less.

When “about” refers to an amino acid residue within the region substituted by CVF (from substitution of C3 amino acid residues into CVF region), the term “about” refers to a range of plus and/or minus 10 amino acids or less for the position. In some embodiments, the term “about” refers to a range of plus and/or minus 5 amino acids or less or 2 amino acids or less.

### Modified Human C3 Proteins and Compositions

The invention provides modified human complement C3 proteins (used interchangeably with “modified human C3 proteins” or “modified C3 proteins”). Amino acid positions indicated herein for the modified human C3 proteins correspond to the amino acid positions in SEQ ID NO:1, but may or may not be the actual position(s) in the modified human C3 protein.

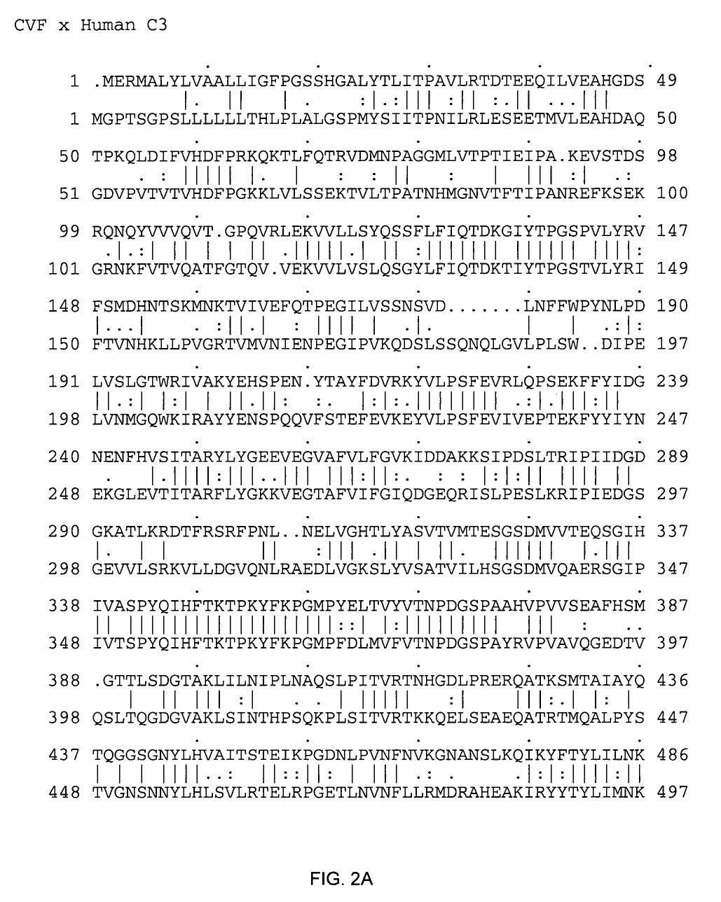

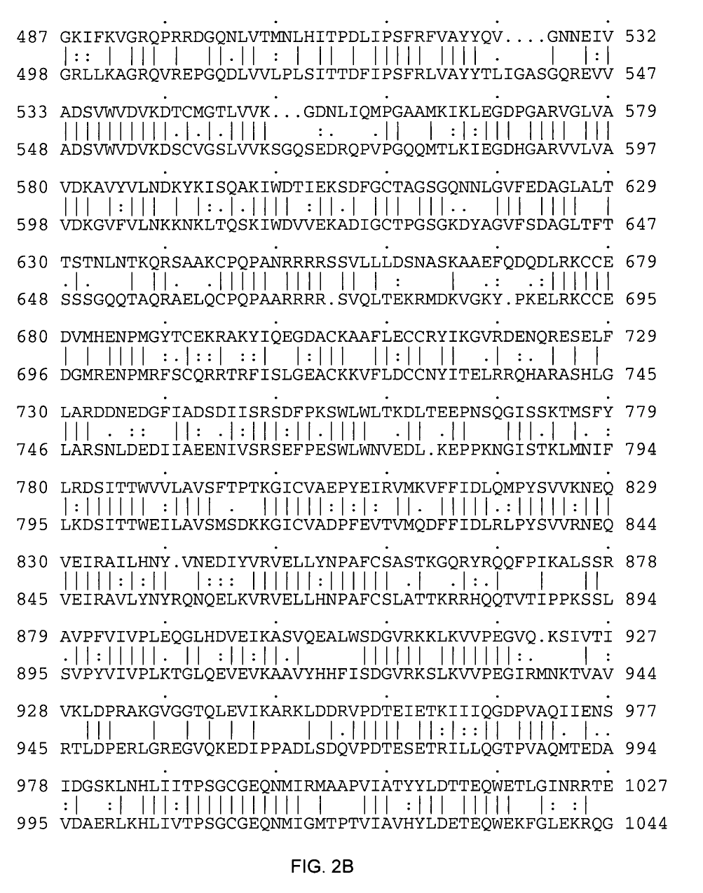

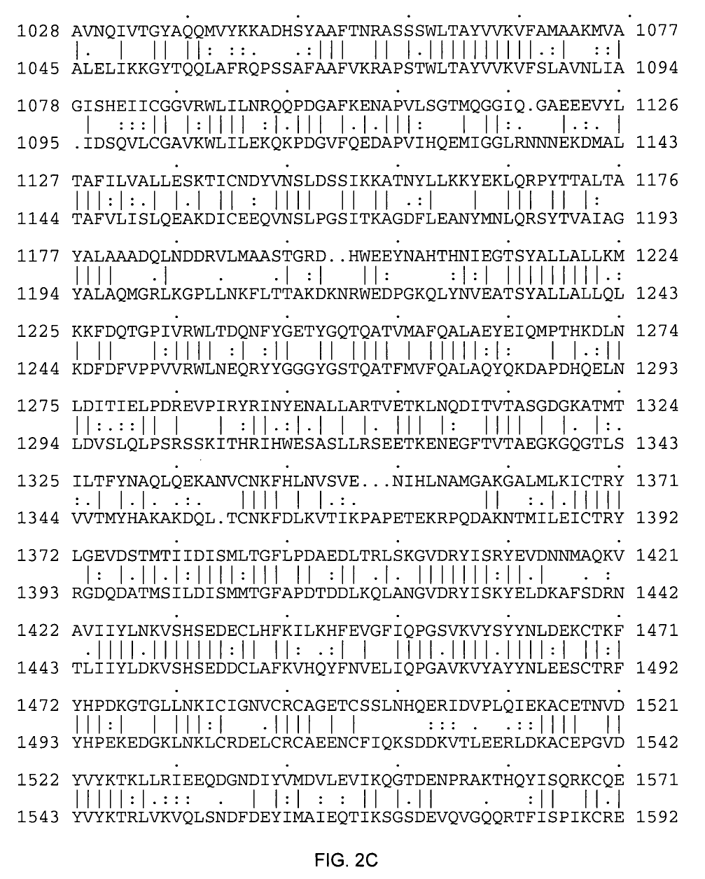

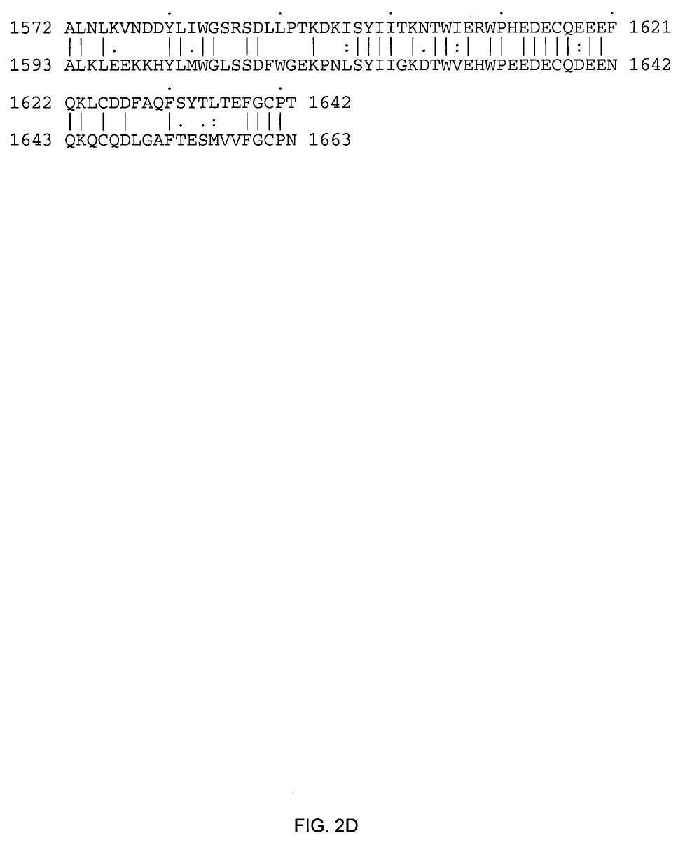

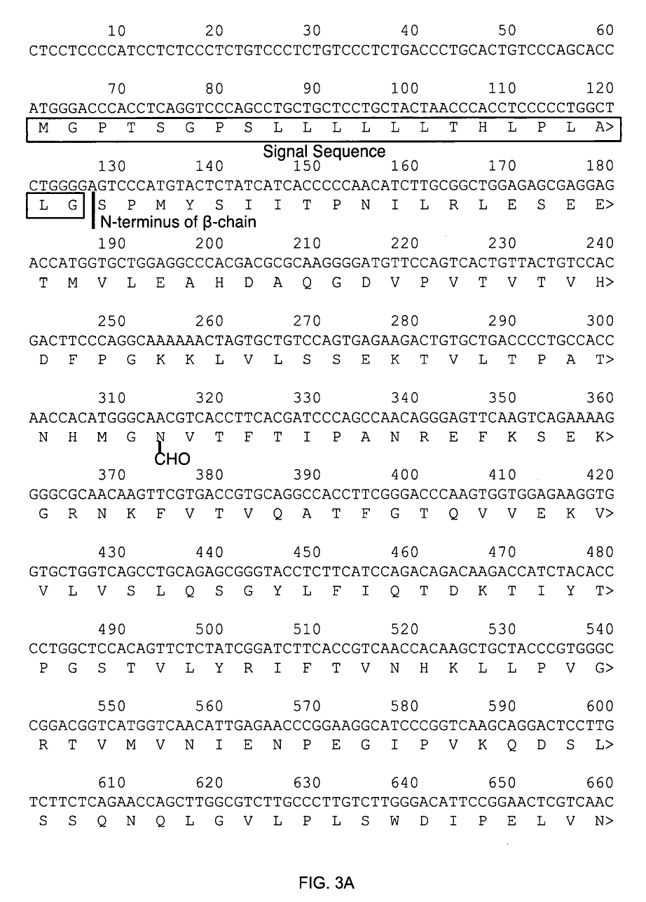

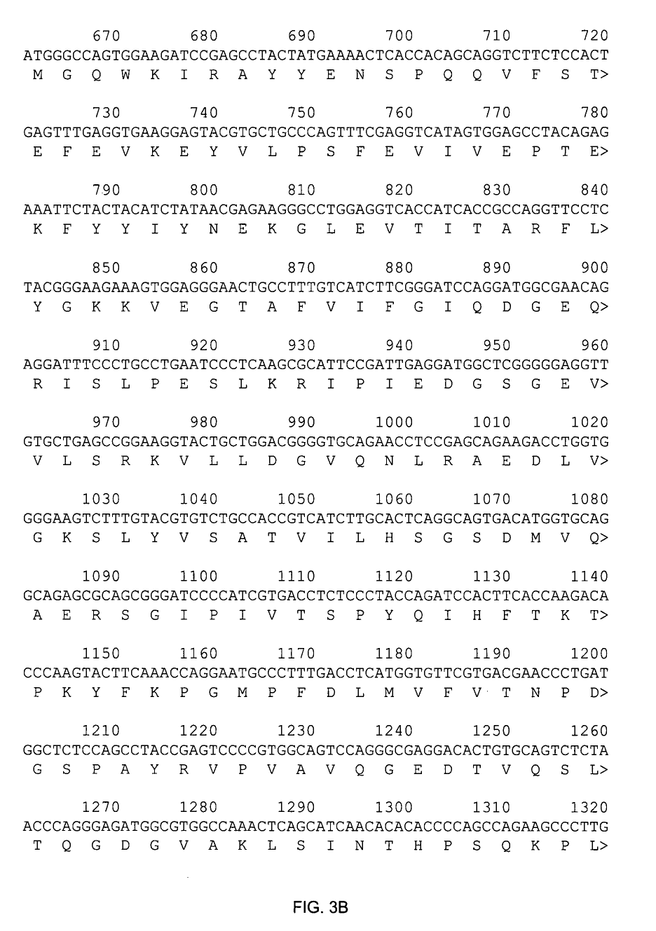

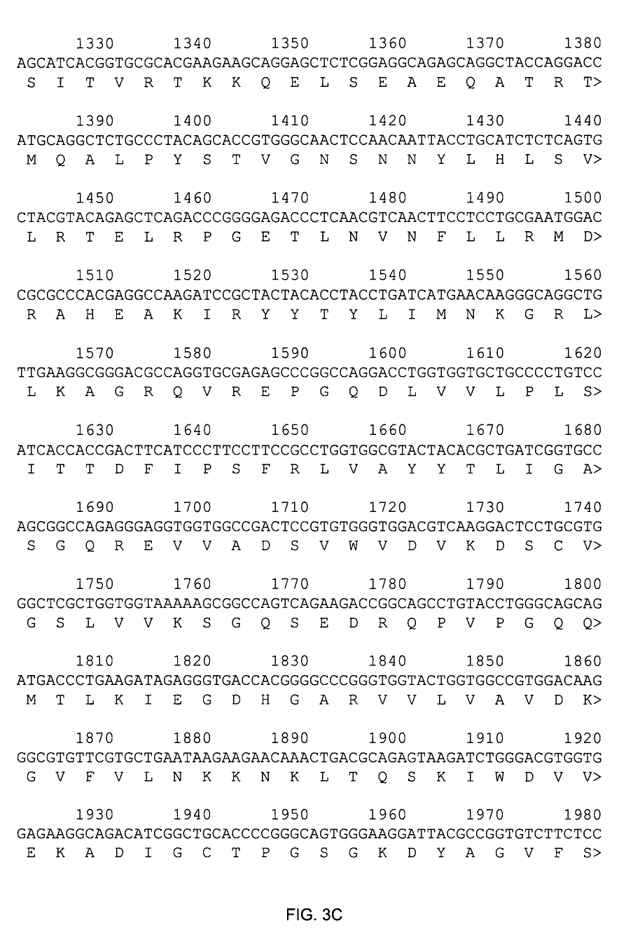

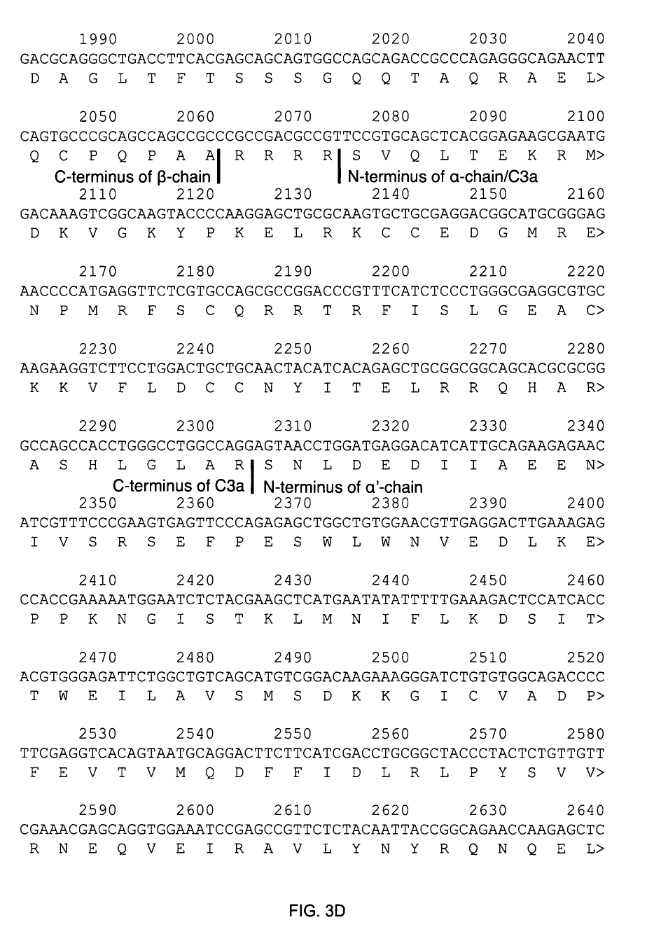

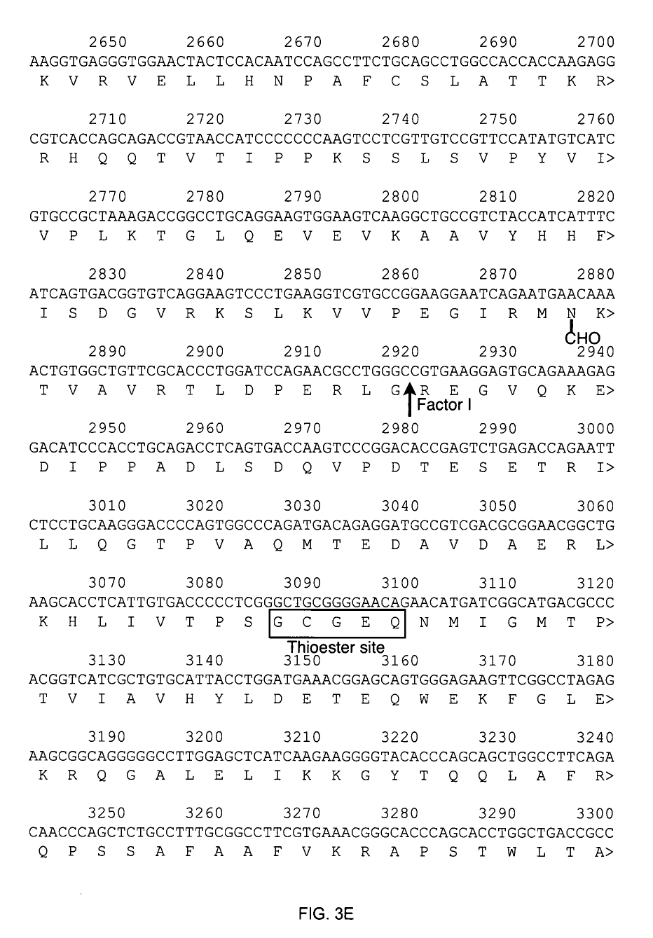

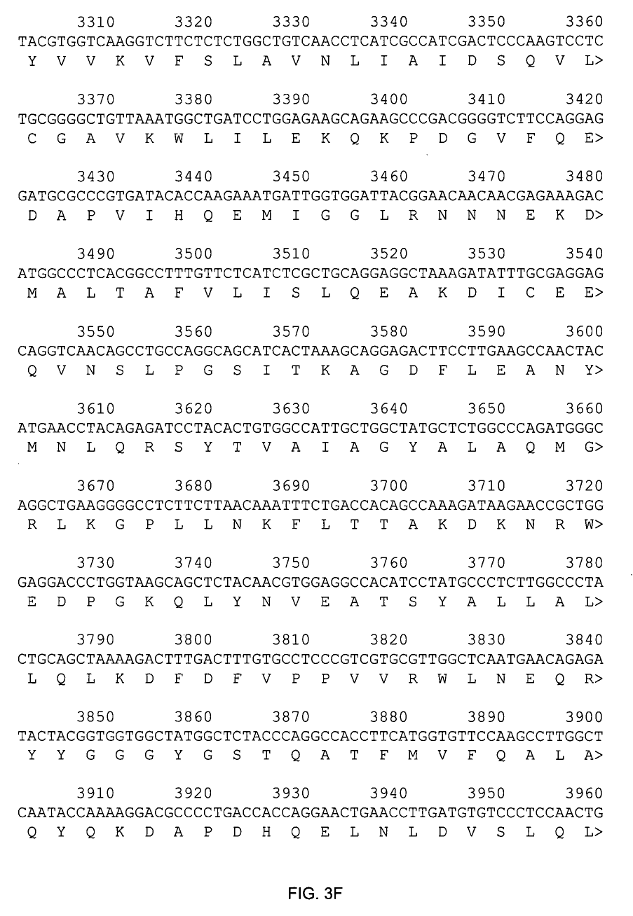

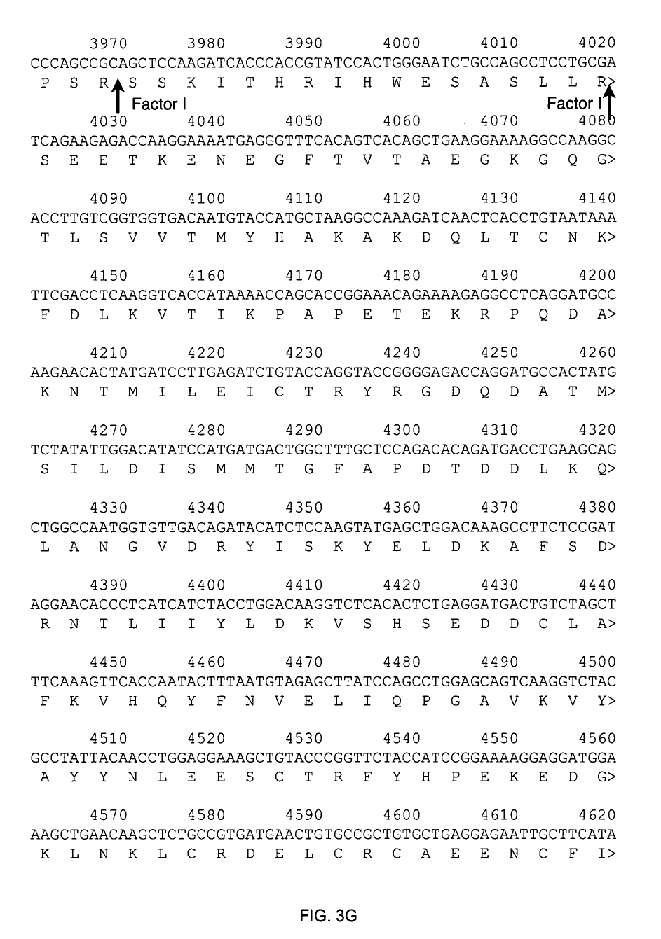

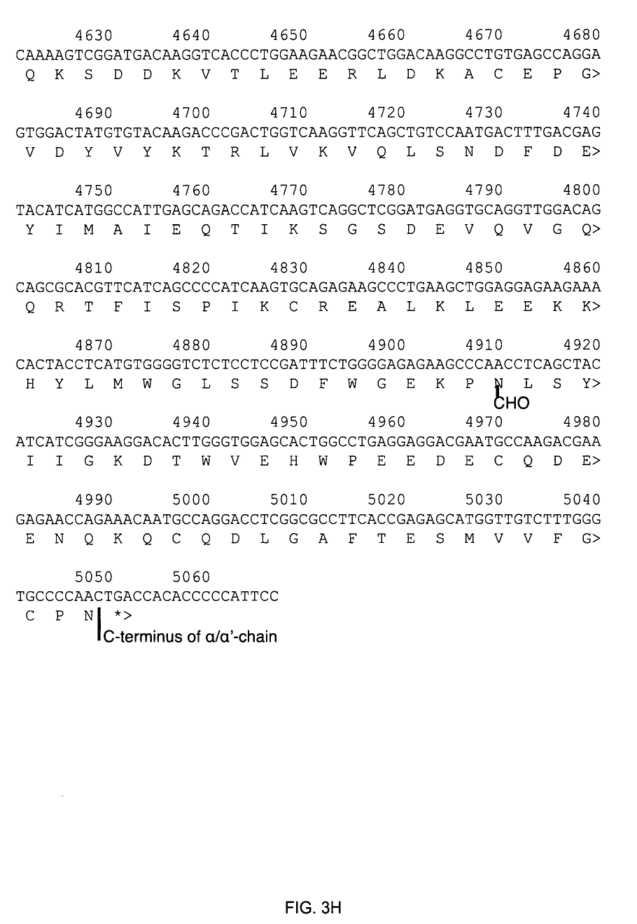

The modified human C3 proteins of the invention comprise a human C3 protein, wherein the amino acid residues in the human C3 protein corresponding to amino acid residues about 1496 to about 1663 of SEQ ID NO:1 (amino acid sequence of pro-human C3) are substituted with a corresponding portion of a Cobra Venom Factor (CVF) protein (for example, amino acid residues about 1475 to about 1642 of SEQ ID NO:2), and wherein one or more amino acid residues in the CVF portion of the modified human complement C3 protein are further modified. The corresponding portion of a CVF protein can be determined by aligning the two amino acid sequences using methods known in the art. For example, an alignment between Naja naja CVF and human C3 protein sequence is shown in FIG. 2.

One or more amino acids in the CVF portion of the modified human complement C3 protein may be modified (such as substituted) in one or more of the following regions: about 1499 to about 1501, about 1507 to about 1510, about 1519 to about 1550, about 1519 to about 1524, about 1528 to about 1532, about 1596 to about 1617, about 1596 to about 1611, about 1598 to about 1600, about 1598 to about 1608, about 1607 to about 1608. In some embodiments, at least one, at least two, at least three, at least four, at least five, at least six, or more amino acids in one or more of these regions are modified (such as substituted). In some embodiments, any of one, two, three, four, five, six or more amino acids in one or more of these regions are modified. In some embodiments, one or more amino acids in the region of amino acid residues 1499-1501 and amino acid residues 1519-1550 are modified (such as substituted). In some embodiments, the amino acids in one or more of these regions are substituted into corresponding amino acids in a human C3 protein, a cobra C3 protein, or other mammalian C3 proteins.

In some embodiments, amino acids in regions such as amino acid residues about 1499 to about 1501, amino acid residues about 1519 to about 1550, amino acid residues about 1598 to about 1600, amino acid residues about 1596 to about 1617, amino acid residues about 1596 to about 1611, amino acid residues about 1598 to about 1608, amino acid residues about 1607 to about 1608, amino acid residues about 1507 to about 1510, amino acid residues about 1519 to about 1524, and amino acid residues about 1528 to about 1532 are substituted with a different amino acid, for example, one or more corresponding amino acids of SEQ ID NO:1 or a conservative substitution thereof. In some embodiments, the whole region is substituted with corresponding amino acids of SEQ ID NO:1.

In some embodiments, the following substitutions may be made: 1) T1499D and L1501K; 2) I1507R, G1508D, N1509E, and V1510L; 3) S1519F, S1520I, L1521Q, N1522K, H1523S, and Q1524D; 4) D1528T, V1529L, P1530E, L1531E, and Q1532R; 5) 1519-1550 replaced with corresponding amino acid residues of SEQ ID NO:1; 6) 1596-1617 replaced with corresponding amino acid residues of SEQ ID NO:1; 7) 1596-1611 replaced with corresponding amino acid residues of SEQ ID NO:1; 8) V1598L, N1599D, D1600N, S1607L, and R1608S; 9) V1598L, N1599D, and D1600N; or 10) S1607L, and R1608S.

The invention also provides a modified human C3 protein, comprising a human C3 protein, wherein amino acid residues in the human C3 protein corresponding to amino acid residues about 1496 to about 1663 of SEQ ID NO:1 are substituted with the corresponding portion of a Cobra Venom Factor (CVF) protein, wherein the CVF portion of the modified human C3 protein comprises a further substitution of one or more amino acid residues in the region of amino acid residues about 1499 to about 1501. In some embodiments, the further substitution of one or more amino acid residues is a substitution with one or more corresponding amino acids of SEQ ID NO:1 or conservative substitutions thereof. In some embodiments, the further substitution of one or more amino acid residues is T1499X and L1501Z, wherein X is amino acid residue D or a conservative substitution thereof and Z is amino acid residue K or a conservative substitution thereof. In some embodiments, the further substitution of one or more amino acid residues is T1499D and L1501K.

The invention also provides a modified human C3 protein, comprising a human C3 protein, wherein amino acid residues in the human C3 protein corresponding to amino acid residues about 1496 to about 1663 of SEQ ID NO:1 are substituted with the corresponding portion of a Cobra Venom Factor (CVF) protein, wherein the CVF portion of the modified human C3 protein comprises a further substitution of one or more amino acid residues in the region of amino acid residues about 1519 to about 1550. In some embodiments, the further substitution of one or more amino acid residues is a substitution with one or more corresponding amino acids of SEQ ID NO:1 or conservative substitutions thereof. In some embodiments, the further substitution of one or more amino acid residues is a substitution with corresponding amino acids about 1519 to about 1550 of SEQ ID NO:1 or conservative substitutions thereof.

In some embodiments, at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, or 32 amino acids of amino acid residues 1519-1550 are substituted. In some embodiments, less than 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 amino acids of amino acid residues 1519-1550 are substituted. In some embodiments, one or more amino acids in one or more of the following regions may be substituted: 1519-1524, 1519-1525, 1525-1530, 1528-1532, 1530-1535, 1535-1540, 1540-1545, or 1545-1550. In some embodiments, at least one, at least two, at least three, at least four, at least five, or at least six amino acids in one or more of these regions are substituted. In some embodiments, any of one, two, three, four, five, or six amino acids in one or more of these regions are substituted. In some embodiments, at least one, at least two, at least three, at least four, at least five, at least six, at least seven, or at least eight of these regions comprise substitutions. In some embodiments, any of one, two, three, four, five, six, seven, or eight regions comprise substitutions.

The invention also provides a modified human C3 protein, comprising a human C3 protein, wherein amino acid residues in the human C3 protein corresponding to amino acid residues about 1496 to about 1663 of SEQ ID NO:1 are substituted with a corresponding portion of a Cobra Venom Factor (CVF) protein, wherein the CVF portion of the modified human C3 protein comprises a further substitution of one or more amino acid residues is in the region of amino acid residues about 1598 to about 1600. In some embodiments, the further substitution of one or more amino acid residues is V1598X, N1599Z1, and D1600Z2, wherein X is amino acid residue L or a conservative substitution thereof, Z1 is amino acid residue D or a conservative substitution thereof, and Z2 is amino acid residue N or a conservative substitution thereof. In some embodiments, the further substitution of one or more amino acid residues is V1598L, N1599D, and D1600N.

The invention also provides modified human complement C3 proteins comprising a human C3 protein, wherein the amino acid residues in the human C3 protein corresponding to amino acid residues 1496 to 1617 of SEQ ID NO:1 are substituted with a corresponding portion of a Cobra Venom Factor (CVF) protein, amino acid residue E at 1654 is substituted with amino acid Y, and amino acid residue V at 1658 is substituted with amino acid E.

Suitable conservative substitutions of amino acids are known to those of skill in this art and generally can be made without altering an activity of a resulting modified human complement C3 protein. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. co., p. 224). Such substitutions can be made, for example, in accordance with those set forth in Table 1 as follows:

| Original Residue | Conservative Substitutions |
|---|---|
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (B) | Asp; Gln |
| Gly (G) | Ala |
| His (H) | Asn; Gln; Lys; Arg |
| Ile (I) | Leu; Val; Met; Ala; Phe; |
| | Norleucine |
| Leu (L) | Norleucine; Ile; Val; Met; |
| | Ala; Phe |
| Lys (K) | Arg; Gln; Asn |
| Met (M) | Leu; Phe; Ile |
| Phe (F) | Leu; Val; Ile; Ala; Tyr |
| Pro (P) | Ala |
| Ser (S) | Thr |
| Thr (T) | Ser |
| Trp (W) | Tyr; Phe |
| Tyr (Y) | Trp; Phe; Thr; Ser |
| Val (V) | Ile; Leu; Met; Phe; Ala; |
| | Norleucine |

Other substitutions, including non-conservative changes, also are permissible and can be determined empirically or in accord with other known conservative or non-conservative substitutions.

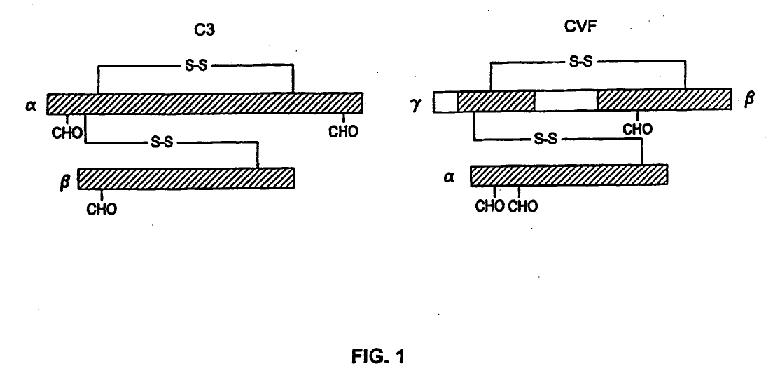

The modified human C3 proteins of the present invention may be in any forms. For example, the modified C3 protein may be in a single chain form comprising the signal sequence or in a single chain form with the signal sequence being cleaved. The modified C3 protein may be in a two-chain form that resembles the structure of human C3 as shown in FIG. 1. The modified human C3 protein may be in a form that resembles human C3b, wherein the C3a portion is cleaved. Depending on host cells that the modified C3 proteins are produced from, the cleavage sites may not be exactly the cleavage site for human C3 proteins. For example, one, two, or more amino acids on either end of the cleavage site of human C3 may be cleaved for the modified C3 protein.

The modified human complement C3 proteins of the present invention have one or more of the following characteristics: (a) ability to deplete complement; (b) ability to mediate the cleavage of factor B; (c) ability to form C3 convertase; (d) ability to cleave C3 and/or C5 upon activation; (e) increased resistance to the regulatory actions of factors H and/or I as compared to native human C3 protein; (f) ability to bind to factor D; (g) increased intrinsic half-life than native human C3 protein; and (h) less immunogenicity than CVF. Methods for assaying these activities are known in the art and some of them are described in the Examples.

The catalytic activity of convertase containing the modified human C3 protein is in some embodiments at least 50% that of the convertase containing CVF, and may be greater than that of the convertase containing native human C3. In some embodiments, the catalytic activity is about any of 60%, 70%, 80% 90% or 100% that of the CVF convertase. In some embodiments, the invention provides convertases containing the modified human C3 protein having a catalytic activity that falls between the two, or that exceeds the activity of the convertase containing native human C3. Thus, the invention additionally provides convertases containing the modified human C3 protein having catalytic activity from about 10% to about 1000%, or more, that of the convertase containing CVF, including but not limited to about any of 20%, 30%, 40%, 50%, 60%, 70%, 80%, 90%, 100%, 110%, 135%, 150%, 200%, 300%, 400%, 500%, 750%, 1000% and more.

Based on the k_{cat}/Kₘ, the catalytic efficiency is approximately eight-fold greater for C3b,Bb compared to CVF,Bb when cleaving C3. In some embodiments, the catalytic efficiency of a convertase containing the modified human C3 protein is at least 50% that of the convertase containing CVF, and may be greater than that of the convertase containing native human C3b. In some embodiments, the invention also provides convertases containing the modified human C3 protein having a catalytic efficiency that falls between the two, or that exceeds the efficiency of the convertase containing native human C3b. Thus, the invention additionally provides convertases containing the modified human C3 protein having catalytic efficiency from about 10% to about 1000%, or more, that of the convertase containing CVF, including but not limited to about any of 20%, 30%, 40%, 50%, 60%, 70%, 80%, 90%, 100%, 110%, 135%, 150%, 200%, 300%, 400%, 500%, 750%, 1000% and more.

The C5 cleaving activity of the modified human C3 proteins may be decreased as compared to native human C3. The C5 cleaving activity can be from non-detectable to about 50% of the activity native human C3, including but not limited to about any of 5%, 10%, 15%, 20%, 25%, 30%, 35%, 40%, 45%, and 50%.

In some embodiments, the binding affinity of the modified human C3 proteins to Factor B and/or its subsequent cleavage by factor D may be reduced. However, as long as at least a functional amount of the catalytic activity and the complement depleting activity remain, the modified human C3 protein is useful.

The invention further provides convertases having the modified human C3 proteins exhibiting substantially the same complement-activating activity of those containing natural CVF. The term “exhibit substantially the same complement-activating activity of natural CVF” means that the modified human C3 protein of the present invention have from about 5% to about 100%, from about 50 to about 97%, from about 80 to about 97% of the level of the complement activating activity of natural CVF as measured by the method of Cochran et al. ((1970) J. Immunol. 105(1)), 55-69).

In some embodiments, the modified human C3 proteins of the present invention have immunogenicity less than that of CVF. In some embodiments, the modified human C3 protein is substantially non-immunogenic. The modified human C3 protein may be as non-immunogenic as C3, or may be at least about any of 50%, 60%, 70%, 80%, 90% less immunogenic than CVF. Immunogenicity may be measured using methods known in the art, for example, methods used for measuring immunogenicity in a human.

In some embodiments, the intrinsic half-life of the convertase formed with the modified human C3 protein is greater than about 1.5 minutes or greater than about 10 minutes. In some embodiments, the intrinsic half-life can fall generally between that of the CVF-containing convertase (7 hours or longer) and that of native human C3 (1.5 minutes), including but not limited to about any of: 2 minutes, 10 minutes, 20 minutes, 30 minutes, 40 minutes, 50 minutes, 60 minutes, 90 minutes, 2 hours, 2.5 hours, 3 hours, 3.5 hours, 4 hours, 4.5 hours, 5 hours, 5.5 hours, 6 hours, 6.5 hours, 7 hours, 8 hours, 9 hours, 10 hours, 10.5 hours, or more. Modified human C3 proteins with short convertase intrinsic half-lives and/or short plasma half-lives and those with long convertase intrinsic half-lives and/or long plasma half-lives may be used for different applications.

In some embodiments, the resistance of the modified human C3 proteins to factors H and/or I is greater than that for native human C3. In some embodiments, the modified human C3 proteins have about the same level of resistance to factors H and/or I as that of CVF. In some embodiments, further modification in other parts of the molecule may be necessary to achieve resistance to factors H and/or I.

The modified human C3 protein of the invention may be linked to another polypeptide or a carrier. For example, the modified human C3 protein may be linked to an antibody or fragments thereof. Methods for making fusion proteins or conjugations are known in the art.

The invention also provides polynucleotides comprising a nucleic acid sequence encoding any of the modified human C3 protein described herein, as well as vectors comprising the polynucleotide and host cells comprising the polynucleotide or containing the vectors. The invention also provides methods of making the vectors and compositions comprising the vectors.

Polynucleotides complementary to any such sequences are also encompassed by the present invention. Polynucleotides may be single-stranded (coding or antisense) or double-stranded, and may be DNA (genomic, cDNA or synthetic) or RNA molecules. RNA molecules include HnRNA molecules, which contain introns and correspond to a DNA molecule in a one-to-one manner, and mRNA molecules, which do not contain introns. Additional coding or non-coding sequences may, but need not, be present within a polynucleotide of the present invention, and a polynucleotide may, but need not, be linked to other molecules and/or support materials. Methods for preparing polynucleotides are known in the art.

The polynucleotide containing the nucleic acid sequence encoding of any of the modified human C3 proteins described herein may be cloned into vectors. Suitable cloning vectors may be constructed according to standard techniques, or may be selected from a large number of cloning vectors available in the art. While the cloning vector selected may vary according to the host cell intended to be used, useful cloning vectors will generally have the ability to self-replicate, may possess a single target for a particular restriction endonuclease, and/or may carry genes for a marker that can be used in selecting clones containing the vector. Suitable examples include plasmids and bacterial viruses, e.g., pUC18, pUC19, Bluescript (e.g., pBS SK+) and its derivatives, mp 18, mp 19, pBR322, pMB9, ColE1, pCR1, RP4, phage DNAs, and shuttle vectors such as pSA3 and pAT28. Examples of vectors for expression in Drosophila cells are pMT/BiP-V5-HisA, pMT/BiP-V5-HisB, and pMT/BiP-V5-HisC. Example of vectors for expression in Pichia pastoris are pPICZA, pPICZB, pPICZC, pPICZα-A, pPICZα-B, and pPICZα-C. These and many other cloning vectors are available from commercial vendors such as BioRad, Strategene, and Invitrogen.

Expression vectors generally are replicable polynucleotide constructs that contain a polynucleotide according to the invention. It is implied that an expression vector must be replicable in the host cells either as episomes or as an integral part of the chromosomal DNA. Suitable expression vectors include but are not limited to plasmids, viral vectors, including adenoviruses, adeno-associated viruses, retroviruses, cosmids, and expression vector(s) disclosed in PCT Publication No. WO 87/04462. Vector components may generally include, but are not limited to, one or more of the following: a signal sequence; an origin of replication; one or more marker genes; suitable transcriptional controlling elements (such as promoters, enhancers and terminator). For expression (i.e., translation), one or more translational controlling elements are also usually required, such as ribosome binding sites, translation initiation sites, and stop codons.

The vectors containing the polynucleotides of interest can be introduced into the host cell by any of a number of appropriate means, including electroporation, transfection employing calcium chloride, rubidium chloride, calcium phosphate, DEAE-dextran, or other substances; microprojectile bombardment; lipofection; and infection (e.g., where the vector is an infectious agent such as vaccinia virus). The choice of introducing vectors or polynucleotides will often depend on features of the host cell.

The invention also provides host cells comprising any of the polynucleotides described herein. Any host cells capable of over-expressing heterologous DNAs can be used for the purpose of isolating the genes encoding the protein of interest. Non-limiting examples of mammalian host cells include but not limited to COS, HeLa, and CHO cells. See also PCT Publication No. WO 87/04462. Suitable non-mammalian host cells include prokaryotes (such as E. coli or B. subtillis) and yeast (such as S. cerevisae, S. pombe, P. pastoris, or K. lactis). Other examples of host cells that can be used are a Drosophila S2 cell, a Sf9 cell, a HiFive cell, a BHK cell, and an HEK293 cell.

A modified human C3 protein may be produced by culturing the host cell comprising the polynucleotides under a condition that the modified human C3 protein is expressed; and purifying the modified human C3 protein. In some embodiments, the modified human C3 protein is purified from the culture medium of the host cell. The invention also provides modified human C3 proteins produced by the method. In some embodiments, the modified C3 protein produced may be in more than one forms (e.g., the protein produced may be a mixture containing both single and double forms).

The invention also provides compositions (including pharmaceutical compositions) comprising any of the modified human C3 proteins. It is understood that the compositions can comprise more than one modified human C3 proteins or modified human C3 protein in more than one forms. The composition used in the present invention can further comprise pharmaceutically acceptable carriers, excipients, or stabilizers (Remington: The Science and practice of Pharmacy 20th Ed. (2000) Lippincott Williams and Wilkins, Ed. K. E. Hoover), in the form of lyophilized formulations or aqueous solutions. Acceptable carriers, excipients, or stabilizers are nontoxic to recipients at the dosages and concentrations, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

The modified C3 proteins described herein may also be formulated for controlled or sustained release. Suitable examples of sustained-release preparations include semipermeable matrices of solid hydrophobic polymers containing modified C3 proteins, which matrices are in the form of shaped articles, e.g. films, or microcapsules. Examples of sustained-release matrices include polyesters, hydrogels (for example, poly(2-hydroxyethyl-methacrylate), or ‘poly(v nylalcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid. Another example of sustained release drug-delivery system that can be used is the ATRIGEL® made by Atrix Laboratories. See, for example U.S. Pat. No. 6,565,874. The ATRIGEL® drug delivery system consists of biodegradable polymers, similar to those used in biodegradable sutures, dissolved in biocompatible carriers. Proteins may be blended into this liquid delivery system at the time of manufacturing or, depending upon the product, may be added later by the physician at the time of use. When the liquid product is injected through a small gauge needle or placed into accessible tissue sites through a cannula, displacement of the carrier with water in the tissue fluids causes the polymer to precipitate to form a solid film or implant. Proteins encapsulated within the implant are then released in a controlled manner as the polymer matrix biodegrades with time. Depending upon the patient's medical needs, the Atrigel system can deliver proteins over a period ranging from days to months. Injectable sustained release systems, such as ProLease®, Medisorb®, manufactured by Alkermes may also be used.

It is understood that aspect and embodiments of the invention described herein include “consisting” and/or “consisting essentially of” aspects and embodiments.

### Therapeutic Use of the Modified Human C3 Proteins

The invention provides methods for the depletion of complement by the modified human complement C3 proteins.

Local treatment may be effected in a number of ways to produce a result of depletion of complement or activation of complement, depending on the desired effect. In one embodiment, local depletion is effected when an effective amount of the modified human C3 proteins are administered locally to an organ, tissue, cavity, or intradermally. This results in a temporary and complete depletion of complement in the area. Local depletion or activation may also be effected using an insulin-type pump that produces an intermittent or constant flow of the modified human C3 protein to a selected site. Alternatively, local activation of complement may employ a specific monoclonal antibody which, when chemically attached to the modified human C3, can localize it to a specific tissue, a disease, or an infected cell to cause continuous activation of complement in that area. In other embodiments, the antibody can be attached to the modified human C3 protein via recombinant DNA technology.

Systemic depletion is effected when an effective amount of the modified human C3 proteins are administered systemically, for example, intravenously or intraperitoneally. This may result in a temporary and complete depletion of complement systemically. This method can be used for reperfusion injury, coronary heart surgery, transplantation and/or systemic disease, particularly during a flare-up of symptoms or during episodical activity.

Depletion of complement can also be effected by an ex vivo treatment, for example, by transfusing circulation of an individual through a matrix bearing the modified human C3 proteins to remove complement. The method may further include a step of removing (e.g., by dialysis) anaphylactic peptides (e.g., C3a and C5a) and other low molecular weight inflammatory mediators (e.g. histamine and nitric oxide) prior to the decomplemented blood (or plasma) being returned to the individual.

Some of the most advantageous qualities of the modified human C3 proteins used in each of these cases and in specific disease states can vary considerably. For example, in those cases for which an immediate, but temporary depletion of complement is desired, a modified protein having a shorter plasma half-life and/or lower stability, but high complement activation activity is preferred. In treating a chronic disease, a long plasma half-life and/or high stability, even if accompanied by a low or sluggish activity, would be preferred. Further, a modified human C3 molecule that does not activate C5 can be particularly advantageous for certain therapies as it prevents the generation of the pro-inflammatory C5a anaphylatoxin.

The modified human C3 proteins described herein may be used for treating or preventing autoimmune diseases, such as, asthma, systemic lupus erythematosus, glomerulonephritis, rheumatoid arthritis, Alzheimer's disease, multiple sclerosis, myocardial ischemia, reperfusion, sepsis, hyperacute rejection, transplant rejection, cardiopulmonary bypass, myocardial infarction, angioplasty, nephritis, dermatomyositis, pemphigoid, spinal cord injury and Parkinson's disease.

The modified human C3 proteins described herein may be used to avoid or ameliorate reperfusion injury in an individual by delivering a modified human C3 protein to the individual, sufficient to deplete complement, before reperfusion in the individual. In some embodiments, the delivering step can include injecting the modified human C3 protein into an artery. In other embodiments, the delivering step can include a local delivery of the modified human C3 protein. In other embodiments, the delivering step can include a systemic delivery of the modified human C3 protein. In some embodiments, reperfusion can include opening a blocked artery. In some embodiments, the reperfusion can occur in connection with transplantation of an organ.

The modified human C3 proteins described herein may be used to increase the efficiency and/or effectiveness of gene therapy by delivering a modified human C3 protein in an amount sufficient to deplete complement before applying gene therapy treatment to the individual.

The modified human C3 proteins described herein may be used to increase delivery of a therapeutic or diagnostic agent by delivering a modified human C3 protein sufficient to increase blood flow before administering the therapeutic or diagnostic agent. In some embodiments, the method can include chemically linking the modified human C3 protein to an antibody with an affinity for a specific tissue prior to the delivering step. In some embodiments, the antibody can be attached to the modified human C3 by recombinant DNA technology. In some embodiments the antibody can be a monoclonal antibody.

The particular dosage regimen, i.e., dose, timing and repetition, will depend on the particular individual and that individual's medical history. Generally, for administration of a modified C3 protein composition, any of the following doses may be used: a dose of at least about 50 mg/kg body weight; at least about 20 mg/kg body weight; at least about 10 mg/kg body weight; at least about 5 mg/kg body weight; at least about 3 mg/kg body weight; at least about 2 mg/kg body weight; at least about 1 mg/kg body weight; at least about 750 μg/kg body weight; at least about 500 μg/kg body weight; at least about 250 ug/kg body weight; at least about 100 μg/kg body weight; at least about 50 μg/kg body weight; at least about 10 ug/kg body weight; at least about 1 μg/kg body weight, or more, is administered. Empirical considerations, such as the half-life, generally will contribute to determination of the dosage. For repeated administrations over several days or longer, depending on the condition, the treatment is sustained until a desired suppression of disease symptoms occurs or until sufficient therapeutic levels are achieved.

In some individuals, more than one dose may be required. Frequency of administration may be determined and adjusted over the course of therapy. For example, frequency of administration may be determined or adjusted based on the type and severity of the disease to be treated, whether the agent is administered for preventive or therapeutic purposes, previous therapy, the patient's clinical history and response to the agent, and the discretion of the attending physician. Typically the clinician will administer the modified C3 protein until a dosage is reached that achieves the desired result. In some cases, sustained continuous release formulations may be appropriate. Various formulations and devices for achieving sustained release are known in the art.

In some embodiments, the invention provides compositions (described herein) for use in any of the methods described herein, whether in the context of use as a medicament and/or use for manufacture of a medicament.

### Kits

The invention also provides kits for use in the instant methods. Kits of the invention include one or more containers comprising a modified human C3 protein described herein. For example, the kit may comprise a modified human C3 protein, comprising a human C3 protein, wherein amino acid residues in the human C3 protein corresponding to amino acid residues 1496 to 1663 of SEQ ID NO:1 (prepro-human C3) are substituted with a corresponding portion of a Cobra Venom Factor (CVF) protein, wherein the CVF portion of the modified human C3 protein comprises a further substitution of one or more amino acid residues in one or more regions including of amino acid residues 1499-1501, amino acid residues 1519-1550, amino acid residues 1598-1600, amino acid residues 1596-1617, amino acid residues 1596-1611, amino acid residues 1598-1608, amino acid residues 1607-1608, amino acid residues 1507-1510, amino acid residues 1519-1524, and amino acid residues 1528-1532.

Kits may further comprise instructions for use in accordance with any of the methods of the invention described herein. Generally, these instructions comprise a description of administration of the modified human C3 protein to deplete complement according to any of the methods described herein. The kit may further comprise a description of selecting an individual suitable for treatment. The instructions relating to the use of a modified human C3 protein may include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The kits of this invention are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). At least one active agent in the composition is a modified human C3 protein. The container may further comprise a second pharmaceutically active agent.

Kits may optionally provide additional components such as buffers and interpretive information. Normally, the kit comprises a container and a label or package insert(s) on or associated with the container.

The following Examples are provided to illustrate but not limit the invention.

### EXAMPLES

### Example 1

### Construction of Modified Human Complement C3 Proteins

The replacement of human C3 sequences with CVF sequences representing important structural features for CVF specific functions allows the creation of modified human C3 proteins with CVF-like functions. FIG. 2 shows the alignment of the amino acid sequences between human C3 (SEQ ID NO:1) and CVF (SEQ ID NO:2). The human C3 molecules in Table 2 and Table 3 are engineered to contain specific CVF sequences so as to create modified human C3 proteins with CVF functions. The amino acid numbering used in the modified human C3 proteins corresponds to the amino acid positions in SEQ ID NO:1, but may or may not be the actual position(s) in the modified human C3 protein.

TABLE 2
Exemplary modified Human C3 constructs

| Construct | |
|---|---|
| Name | Description of Construct |
| HC3-1496 | 1496-1663 CVF |
| HC3-1496-2 | 1496-1663 CVF with 2 substitutions from |
| | human C3, T1499D and L1501K |
| HC3-1496-8 | 1496-1663 CVF with 1519-1550 replaced with |
| | human C3 |
| HC3-1496-9 | 1496-1663 CVF with 5 substitutions from |
| | human C3, Q1571S, T1573S, N1576V, |
| | P1577Q, and R1578V |
| HC3-1496-10 | 1496-1663 CVF with 1596-1617 replaced with |
| | human C3 |
| HC3-1496-11 | 1496-1663 CVF with 1596-1611 replaced with |
| | human C3 |
| HC3-1496-12 | 1496-1663 CVF with 5 substitutions, V1598L, |
| | N1599D, D1600N, S1607L, and R1608S |
| HC3-1496-13 | 1496-1663 CVF with 3 substitutions, V1598L, |
| | N1599D, and D1600N |
| HC3-1496-14 | 1496-1663 CVF with 2 substitutions, S1607L, |
| | and R1608S |
| HC3-1496-15 | 1496-1663 CVF with 4 substitutions from |
| | human C3, I1507R, G1508D, N1509E, and |
| | V1510L |
| HC3-1496-16 | 1496-1663 CVF with 6 substitutions from |
| | human C3, S1519F, S1520I, L1521Q, |
| | N1522K, H1523S, and Q1524D |
| HC3-1496-17 | 1496-1663 CVF with 5 substitutions from |
| | human C3, D1528T, V1529L, P1530E, |
| | L1531E, and Q1532R |

TABLE 3
Exemplary modified Human C3 constructs

| Construct | |
|---|---|
| Name | Description of Construct |
| HC3-1496/ | 1496-1617 CVF |
| 1617 | |
| HC3-1496-3 | 1496-1617 CVF with 1 substitution, E1633H |
| HC3-1496-4 | 1496-1617 CVF with 2 substitutions, E1654Y |
| | and V1658E |

The modified human C3 proteins (also called hybrid proteins or chimerae) were produced by site-directed mutagenesis as described below. Briefly, for site-directed mutagenesis replacing small portions of human C3 sequence with CVF, the procedure of Ho et al. was used (Ho, S. N., Hunt, H. D., Horton, R. M., Pullen, J. K. and Pease, L. R. (1989) “Site-Directed Mutagenesis by Overlap Extension Using the Polymerase Chain Reaction” Gene, 77:51-59) (also called herein overlap extension method). In this method, two PCR reactions were performed, one with a forward primer somewhere upstream from the site of the desired mutation. The second, reverse primer contained the mutation. The second PCR of this round had a forward primer containing the desired mutation, with the reverse primer being downstream from the mutation site. At least one unique restriction site was preferably present in each of the PCR products from this step so that it was possible to transfer the modified DNA back into the original clone. Amplification was done using a large amount of template DNA, and a low number of cycles to minimize mutations introduced by the PCR process.

After amplification, the two products were purified by gel electrophoresis and were isolated from the gel using a Qiaquick Gel extraction kit from Qiagen. Then, the fragments were combined, and another PCR reaction was performed, using the two fragments as the template, and the outside primers as the amplification primers. Again, the PCR reaction was performed using a high concentration of template DNA and only cycled for a few cycles to minimize PCR-caused mutations. The resulting PCR product was cut with the two unique restriction enzymes, size purified on an agarose gel, and the fragment of interest was isolated using the Qiaquick column. The fragments were then cloned into either pBS-HuC3 or pHC3-1550(-sig) that is cut with the same enzymes.

The HC3-1496 was prepared according to the method described in PCT/US2005/05119. Briefly, the plasmid for the production of the modified human C3 protein, HC3-1496, was produced as follows. Two PCR reactions were performed to obtain the human C3 and CVF portions of the coding sequence. In the first, pBS-HuC3-2 was used as a template, with the following oligonucleotides used as primers: HC3H5-3-F1 TCTGTGTGGCAGACCCCTTCGAGG and HC3H5-4-R1 GAGAAGGCCTGTTCCTTTATCCGGATGGTAGAACCGGGTAC. The second PCR used pCVF-FL3Δ as a template, and the following oligonucleotides as primers: HuCC3H5-4-F2 CCGGTTCTACCATCCGGATAAAGGAACAGGCCTTC, and HuC3H5-3-R2 CATCCATGACATAGATATCATTACCATCTTG. The resulting two PCR products were joined in a PCR reaction, using HuC3H5-3-F1 and HuC3H5-3-R2 as primers, and the two PCR fragments as the template. After the second PCR reaction, the product was purified using a Qiagen PCR cleanup kit. It was then cut with NspV, and cloned into pHC3-1550(-sig) that had been cut with the same enzyme and been Calf intestine alkaline phosphate treated. The resulting plasmid was called pHC3-1496. The insert from this plasmid was then isolated as described above, and cloned into pMT-Bip/V5-HisA (Invitrogen) as described above. This plasmid was called pMB/HC3-1496. The modified C3 protein (HC3-1496) encoded by this plasmid was expressed in Drosophila cells, the protein generated had 1) about 20 to 50% complement depletion activity as compared to CVF, 2) factor B cleavage activity about equal to CVF, 3) convertase stability about equal to CVF, 4) C3 cleavage activity about five times better than CVF, and 5) none detected C5 cleavage activity.

In constructing HC3-1496-2, two substitutions were made, T1499D and L1501K, to HC3-1496. To accomplish this, the first round of PCR reactions both used pHC3-1496 as a template. In the first PCR reaction, the primers were HC3-1496-1F(2) (GCGAGGACACTGTGCAGTCTCTAAC) and HC3-1496(2)-1R (CATATCTTATTGAGCTTGCCTTCTCCTTTATCCGG). For the second PCR, the primers used were HC3-1496(2)-2F (CCGGATAAAGGAGAAGGCAAGCTCAATAAGATATG) and HC3-1496-2R(2) (GCTCGTGGATTTTCGTCAGTACCTTGTTTAATAAC). After the PCR reaction, the two fragments were purified using the Qiagen PCR purification kit, and joined together in a PCR reaction containing the two amplicons as template and the outer primers (HC3-1496-1 F(2) and HC3-1496-2R(2)) as primers. Following this PCR reaction, the PCR amplification product was purified using the Qiagen PCR purification kit and cut with BstBI, or its isoschizomer, NspV. The digestion reaction was run on a 1% agarose gel, and the 2.1 kb fragment isolated and purified using the Qiagen gel isolation kit. This fragment was then ligated into the plasmid pHC3-1496 that had been cut with the same enzyme, and CIP treated, and the ligation reactions transformed into E. coli DH5α. Cells containing plasmid were selected on ampicillin plates, and were then grown in LB broth containing ampicillin to obtain miniprep DNA. This DNA was cut with EcoRI to ascertain that the insert was in the correct orientation, and sequenced to ascertain the presence of the desired mutation and absence of any PCR-induced mutations. A midiprep (50 ml growth) of one clone was prepared so that the insert could be cloned into the expression vector. It was given the name pHC3-1496-2. The open-reading frame DNA from pHC3-1496-2 was isolated by cleavage of the plasmid with AfeI, NotI, and DraI (to fragment the vector portion of the plasmid to allow easier isolation of the insert). The digest was run on an agarose gel, and the ˜5 kb band isolated as described above. This was ligated into pMT/BiP-V5-HisA that had been cut with EcoRV and NotI, and the ligation mixture used to transform competent E. coli DH5α cells. DNA from positive clones was digested with BamHI to determine that the plasmid contained the proper insert, and the ends of the insert were sequenced to make sure the fragment was cloned in-frame. The resulting plasmid was called pMB/HC3-1496-2.

HC3-1496-8 is HC3-1496 with human C3 sequences replacing the CVF sequences between position 1519 and 1550. The overlap extension method using CVF and human C3 sequences was utilized in the construction of the expression vector for this modified human C3 protein. For the first round of PCR reactions, the first PCR used pHC3-1496 as a template, and HC3-1496-1F(2) and HC3-1496(1504-1550)-1R (CATCCGACTTTTGTATGAAACAGGTTTCTCCTGCAC) as primers, while the second reaction used pHC3-1550 as the template and HC3-1496(1504-1550)-2F (GTGCAGGAGAAACCTGTTTCATACAAAAGTCGGATG) and HC3-1496-2R(2) as primers. Following the PCR reaction, the fragments were purified as described above, and joined together in the overlap extension PCR, using HC3-1496-1F(2) and HC3-1496-2R(2) as the primers. The PCR product was purified as described above, cut with BstBI, and cloned into pHC3-1550(-sig) that had been cut with the same enzyme and CIP treated. The orientation of the insert was determined by EcoRI digestion, and DNA from clones with the insert in the correct orientation were sequenced to ascertain that they have the desired insert without any PCR-induced mutations. One colony that contained a plasmid with the correct sequence was grown up, and the plasmid, called pHC3-1496-8, isolated. The coding portion of this plasmid was isolated and cloned into pMT/BiP-V5-HisA as described above. The final plasmid was called pMB-HC3-1496-8. The modified C3 protein (HC3-1496-8) encoded by this plasmid was expressed in Drosophila cells. The protein produced had been tested for various properties as described in Example 3. This protein was much less active in complement depletion than HC3-1496, had very rapid factor B cleavage activity, appeared to have short half-life for convertase stability, and were at least two times more active on C3 cleavage than HC3-1496.

HC3-1496-9 is a modified human C3 protein that contained the following human C3 substitutions in the CVF portion of the protein: Q1571S, T1573S, P1577Q and R1578V. The expression plasmid for HC3-1496-9 was prepared in a manner similar to that described for HC3-1496-2. Again, for the first round of PCR reactions, two reactions were set up, both using pHC3-1496 as a template. The first reaction used the following primers: HC3-1496-9-F1 (TGGTGGATTACGGAACAACAACGAG) and HC3-1496-9-R1 (ACCTCATCCGAGCCTGATTTAATAACTTC), while the second used: HC3-1496-9-F2 (GCTCGGATGAGGTGCAGGTTGCAAAGACCCAC) and HC3-1496-9-R2 (AAGGAAGGGAAGAAAGCGAAAGGAG). Following the initial PCR reactions, the fragments were purified and used as templates in the second round of PCR, using HC3-1496-9-F1 and HC3-1496-9-R2 as primers. The resulting amplicon was purified, cut with SfiI and NotI, gel purified and ligated into pHC3-1496 that had been cut with the same two enzymes. Miniprep DNA was sequenced to ascertain that the plasmid contained the correct insert. Midiprep DNA of one clone was prepared. It was called pHC3-1496-9. The insert was excised from this plasmid and cloned into pMT/BiP-V5-HisA as described above. The resulting plasmid was called pMB-HC3-1496-9.

HC3-1496-10 is a modified human C3 protein in which the CVF sequences from residue 1596-1617 have been replaced with human C3 sequences. Since both HC3-1496-10 and HC3-1496-11 contain a human C3 substitution in the middle of the CVF portion of HC3-1496, it was necessary to first prepare an intermediate plasmid, pHC3-1496/1596, before the final constructions could be performed. To prepare the first plasmid, the overlap extension method described above was used to join portions of CVF and C3 DNA. For the first set of PCR reactions, pHC3-1496 was used as the template for the first reaction, with HC3-1496-9-F1 and HC3-1596-1R (CTTCTCCTCCAGATTCAGAGCCTCCTG) as the primers. The second reaction used pBS-HuC3-2 (a plasmid containing the entire coding sequence of human C3) as the template, and HC3-1596-2F (CAGGAGGCTCTGAATCTGGAGGAGAAG) and HC3-1496-9-R2 as the primers. The amplified DNA from these two reactions were purified and joined together as described above, using HC3-1496-9-1F and HC3-1496-9-R2 and primers. The amplified DNA was purified, cut with SfiI and NotI, and ligated into pHC3-1496 that had been cut with the same enzymes. The ligation mixture was transformed into DH5α cells, and DNA from the resulting colonies analyzed by sequencing. DNA was prepared from one clone with the correct sequence and was used for the final construction of pHC3-1496-10 and pHC3-11. This plasmid was called pHC3-1496/1596, and codes for a protein that contains CVF sequences from residue 1496 to 1596.

To obtain the final HC3-1496-10 construct, CVF sequences replaced the C3 sequences from 1617 to the C-terminus by the overlap extension method. The first reaction of the first set of PCR reactions used pHC3-1496/1596 as a template, and used the following primers: HC3-1496-9-F1 and HC3-1496-10-R1 (GTAATGATGTAGGAAATGTTGGGCTTCTCTCC). The second PCR used pHC3-1496 as a template, with the following primers: HC3-1496-10-2F (GGAGAGAAGCCCAACATTTCCTACATCATTAC) and HC3-1496-9-R2. Following the first PCR, the amplified DNA was purified as described above and used as the template in a second, overlap extension reaction, using HC3-1496-9-F1 and HC3-1496-9-R2 as primers. The product of this reaction was purified, cut with SfiI and NotI, gel purified and cloned into pHC3-1496 that had been cut with the same enzymes. After verification of the sequence, one clone, called pHC3-1496-10, was picked for plasmid isolation. The coding sequence from this plasmid was excised from the plasmid and cloned into pMT/BiP-V5-HisA as described above. The final construct was called pMB-HC3-1496-10.

HC3-1496-11 is a protein that is similar to HC3-1496, but contains human C3 sequences from residue 1597 to 1611. The expression plasmid for this protein was prepared in an identical way as the plasmid for HC3-1496-10, except that, in the first set of PCR reactions, the template for the first PCR was pHC3-1496/1596 and the primers used were HC3-1496-9-1F and HC3-1496-11-1R (GAAATTTTATCTTTCGTGGGCAAGAAATCGGAGGAGAG), while the template for the second PCR was pHC3-1496, and the primers were HC3-1496-11-2F (GGTCTCTCCTCCGATTTCTTGCCCACGAAAGATAAAATTTC) and HC3-1496-9-R2. The rest of the construction was performed identically to that of HC3-1496-10. The expression plasmid for this protein is called pMB-HC3-1496-11.

HC3-1496-12 is a modified human C3 protein in which five amino acid residues from the CVF portion of HC3-1496 have been replaced with sequences from cobra C3. The substitutions were: V1598L, N1599D, D1600N, S1607L, and R1608S. The changes were introduced using the site-directed mutagenesis by overlap extension method. For the first round of PCR reactions, both reactions used pHC3-1496 as the template. The primers for the first reaction were: HC3-1496-1F(2) and HC3-1496-12-1R (CCCCAGATCAGATAATCATTATCCAGCTTC), while the second reaction used HC3-1496-12-2F (ATTATCTGATCTGGGGTCTCAGCAGTGACC). Following the PCR reaction, the DNA products were purified as described above, and joined in the overlap extension step, using HC3-1496-1F(2) and HC3-1496-2R(2) as primers. This product was purified, cut with SfiI and NotI, gel purified, and ligated into pHC3-1496 that had been cut with the same enzymes. After transformation, colonies were grown up to prepare plasmid DNA, and plasmids were analyzed by sequence analysis to ascertain that they contained the correct insert without any PCR-induced mutations. One colony containing the correct plasmid, called pHC3-1496-12, was grown up, plasmid DNA isolated and the insert excised and cloned into the expression plasmid pMT/BiP-V5-HisA as described above. The final expression plasmid was called pMB-HC3-1496-12.

HC3-1496-13 and HC3-1496-14 are modified human C3 proteins based on HC3-1496, containing the first three and the last two amino acid substitutions from HC3-1496-12, respectively. The construction of these two plasmids was performed in a nearly identical manner to that described for HC3-1496-12, with the only difference being the primers used to make the amino acid substitutions. For HC3-1496-13, the two mutation-inducing primers are: HC3-1496-13-1R (CAGATAATCATTATGGAGCTTCAGATTCAG) and HC3-1496-13-2F (CTGAAGCTGGATAATGATTATCTGATCTGG). For HC3-1496-14, the two primers are: HC3-1496-14-1F (GGCAACAGGTCACTGCTGAGACC) and HC3-1496-14-2F (GGGTCTCAGCAGTGACCTGTTGC). The expression plasmids for these two proteins were called pMB-HC3-1496-13 and pMB-HC3-1496-14, respectively.

HC3-1496-15 is a modified version of HC3-1496, in which the following amino acid residues in the CVF portion of the modified human C3 protein were replaced with human sequences: I1507R, G1508D, N1509E, and V1510L. For the first set of PCR reactions, the template was pHC3-1496, and the primers for the first reaction were HC3-1496-1F(2) and HC3-1496-15-1R (GGCACAGTTCATCACGGCATATCTTATTG), while the primers for the second reaction were HC3-1496-15-2F (ATGCCGTGATGAACTGTGCCGATGTG). The reaction products were purified as described above, and joined using the overlap extension PCR reaction as described, with HC3-1496-1F(2) and HC3-1496-2R(2), as the primers. The product from this reaction was purified, cut with BstBI and gel purified as described above. It was then ligated into pHC3-1550(-sig) that had been cut with the same enzymes and CIP treated to prevent self-ligation. After transformation into E. coli DH5α, plasmid DNA was analyzed by EcoRI digestion to determine the orientation of the BstBI insert. Plasmids with the insert in the correct orientation were sequenced to ascertain the presence of the desired mutations and absence of PCR-induced mutations. One colony containing a plasmid with the correct sequence was grown up, and the plasmid, called pHC3-1496-15, isolated. The portion of the plasmid coding for HC3-1496-15 was excised and cloned into the expression vector, pMT/BiP-V5-HisA as described above. The expression plasmid was called pMB-HC3-1496-15.

HC3-1496-16 is a modified human C3 protein with the following human C3 substitutions in the CVF portion of the protein: S1519F, S1520I, L1521Q, N1522K, H1523S, and Q1524D. The construction of this plasmid was performed in a nearly identical manner as the construction of pMB-HC3-1496-15. The only difference is that the primers used for the first round of PCR reactions were HC3-1496-1F(2) and HC3-1496-16-1R (GTCATCCGACTTTTGTATGAAACAGGTTTCTC) and the primers used in the second reaction were HC3-1496-16-2F (CCTGTTTCATACAAAAGTCGGATGAAAGGATTGATG) and HC3-1496-2R(2).

HC3-1496-17 is a modified human C3 protein with the following human C3 substitutions in the CVF portion of the protein: D1528T, V1529L, P1530E, and L1531E. The construction of this plasmid was performed in a nearly identical manner as that of the expression plasmid for HC3-1496-15, with the exception that, in the first round of PCR reactions, the primers used were as follows: in the first PCR, the primers used were HC3-1496-1F(2) and HC3-1496-17-1R(CCGTTCTTCCAGGGTAATCCTTTCCTGATG); in the second PCR reaction, the primers used were HC3-1496-17-2F (ACCCTGGAAGAACGGATTGAAAAAGCCTG) and HC3-1496-2R(2). The expression plasmid was called pMB-HC3-1496-17.

To prepare a plasmid for the expression of HC3-1496/1617, the internal 2.1 kb BstBI fragment was excised from pHC3-1496, and cloned into pHC3-1550/1617 that had been cut with the same enzymes and CIP treated. The orientation of the insert was determined by digestion of the plasmid with EcoRI. Following confirmatory sequencing, one clone was grown up and called pHC3-1496/1617. The insert was excised from this plasmid and cloned into pMT/Bip-V5-HisA as described above. The expression plasmid for HC3-1550/1617 was prepared by using the overlap extension method of site-directed mutagenesis of Ho and coworkers to join portions of CVF and human C3 sequences. The plasmid for expressing HC3-1550/1617 was based on pHC3-1550(-sig). As a first step, two PCR reactions were performed. In the first, pHC3-1550(-sig) was used as a template, with the following primers: HuC3H5-2-F1 (GGATGCCACTATGTCTATATTGGACATATCC) and HuC3H5-2R1 (CCCGATGATGTAGCTGAGTTTATCTTTCGTGGG). The second PCR used pBS-HuC3-2 as a template and HuC3H5-2-F2 (CCCACGAAAGATAAACTCAGCTACATCATCGGG) and HuC3H5-2-R2(2) (AATTGGAGCTCCACCGCGGTGG) as primers. Following the PCR reaction, the amplified DNA was purified using a Qiagen PCR Purification kit, and joined in an overlap extension PCR, using the two fragments as template, and HuC3H5-2-F1 and HuC3H5-2-R2(2) as primers. This fragment was isolated as described above, cut with BsrGI and NotI, and ligated into pHC3-1550(-sig) that had been cut with the same enzymes. Following sequence confirmation, a large preparation of this plasmid (pHC3-1550/1617) was performed, and the protein-coding insert was excised by digestion with AfeI, NotI and DraI (to digest the vector portion of the plasmid). The coding fragment was gel purified using the Qiagen Gel Extraction kit, and cloned into pMT/Bip-V5-HisA that had been cut with EcoRV and NotI. This plasmid was called pMB/HC3-1550/1617.

The protein encoded by HC3-1496/1617 was expressed in Drosophila cells. The protein produced had 1) none or very poor complement depletion activity, 2) rapid factor B cleavage activity, 3) very poor convertase stability, 4) active C3 cleavage activity but convertase formed apparently dissociated rapidly, and 5) none detected C5 cleavage.

HC3-1496-3 is a variation on HC3-1496/1617, described above, in which glutamic acid 1633 has been replaced with a histidine, the residue found in CVF. The expression plasmid for this protein was constructed in a two-stage process. First, a plasmid was constructed that coded for CVF sequences from position 1550 to 1617, with a single amino acid change at position 1633. Then, this plasmid was cut with BstBI, and the larger fragment was isolated. The plasmid pHC3-1496 was cut with the same enzyme, and the 2.1 kb fragment, containing the sequences coding for the HC3-1496 from residue 840 to 1550 was isolated and ligated into the plasmid containing the desired downstream mutation.

To prepare the first plasmid, called pHC3-1550-2, that would ultimately result in HC3-1496-3, the site-directed mutagenesis by overlap extension method of Ho and coworkers was used. In the first round of PCR reactions, the template was pHC3-1550/1617, and the primers used for the first PCR were HuC3H5-F1 (GGATGCCACTATGTCTATATTGGACATATCC) and HC3-1550-1R (GCATTCGTCCTCGTGAGGCCAGTGC) and for the second PCR, HC3-1550-2-2F (GCACTGGCCTCACGAGGACGAATGC) and HuC3H5-2-2R(2) (GCGTAATACGACTCACTATAGGGCGAATTG). Following the first round of PCR reactions, the reaction products were purified as described previously and joined in the overlap-extension step, using HuC3H5-F1 and HuC3H5-2-2R(2) as the primers. The reaction product was purified, cut with BsrGI and NotI, and ligated into pHC3-1550(-sig) that had been cut with the same enzymes. DNA from transformants was sequenced to ascertain that they contained the expected sequence and did not contain any PCR-induced mutations. This plasmid, called pHC3-1550-2, was grown up, and cut with BstBI and treated with CIP to reduce religation of the plasmid. The plasmid pHC3-1496 was also cut with BstBI, and the 2.1 kb plasmid gel isolated as described above. The two fragments were ligated together and transformed into E. coli DH5α. Transformants were screened for the proper orientation of the insert by EcoRI cleavage, and clones containing the insert in the correct orientation were sequenced to ascertain they contained the correct insert in the proper reading frame. The open reading frame of this plasmid, called pHC3-1496-3, was excised and cloned into pMT/BiP-V5-HisA as described above. The final expression plasmid was called pMB-HC3-1496-3. The protein encoded by pMB-HC3-1496-3 was expressed in Drosophila cells. The protein (HC3-1496-3) produced had none or very poor complement depletion activity, rapid factor B cleavage activity, and very poor convertase stability.

HC3-1496-4 is a variation of HC3-1496/1617, in which two changes were made: E1654Y and V1658E. The construction of the expression plasmid was done in a similar manner to that of HC3-1696-3, with the exception that the primers used in the first round of PCR reactions were HuC3H5-F1 and HC3-1550-3-1R (CCCAAACTCAACCATGCTGTAGGTGAAGGC) for the first PCR reaction, and HC3-1550-3-2F (GCCTTCACCTACAGCATGGTTGAGTTTGGG) for the second PCR. The final expression plasmid was called pMB-HC3-1496-4.

### Example 2

### Expression of Modified Human C3 Proteins

The modified C3 proteins are produced in the Drosophila S2 cell system, using the Drosophila Bip signal sequence for secretion of the proteins. Briefly, the plasmids are transfected into Drosophila S2 cells using the calcium phosphate method of Chen and Okayama (Chen, C., and Okayama, H. (1987) Mol. Cell. Biol. 7(8), 2745-2752). S2 cells are transfected with a mixture of expression plasmid and pCoBlast, using a ratio of 19:1 (w:w). Following transfection, cells containing both plasmids are selected using blasticidin (25 μg/ml). For expression, 1-liter cultures of transfected cells are grown in serum-free medium (Hi-Five plus L-glutamine), in the absence of blasticidin. When the cells reach a density of 5×10⁶ cells/ml, production of the recombinant proteins was induced by the addition of CuSO₄ to a final concentration of 25 μM. Cultures are allowed to express recombinant proteins for 4-5 days. Modified human C3 proteins are then purified from the media by a combination of ANX, Sephacryl H-300, and CM-FF chromatography.

Because of the method of cloning of the C3 gene, there are several amino acids (approximately 19) coded for by the cloning vector, that are N-terminal to the N-terminus of human C3. These extra amino acids do not affect the activity of the protein, so they can be removed with no ill effect. In some cases, it can be preferred for there to be at least two amino acids that are an artifact of the restriction sites needed to clone the final construct into the expression vector. In various embodiments, a variety of signal sequences can be used, including the native signal sequence of human C3, as well as any other signal sequence effective in directing entry of the nascent polypeptide into the endoplasmic reticulum.

Other expression systems that can be used, include but are not limited to: Baculovirus infection of Sf9 or HiFive cells (other insect expression systems), CHO cells, COS-7 cells (mammalian expression systems), E. coli, BHK, HEK293 cells, and various yeast expression systems, including the Hansuela yeast expression system.

### Example 3

### Activity Measurements of the Modified Human Complement C3 Proteins

Various modified human C3 proteins are useful for different diseases, and methods of treatment. Thus, it is useful to analyze the functional qualities of the modified human C3 proteins of embodiments of the invention and to use them accordingly. The following methods are employed to analyze the function of purified modified human C3 proteins produced as described in Example 2. The methods described herein, as well as others that are known to those of skill in the art, may be used.

Assays to determine convertase activity. In addition to the specific assays as mentioned below, two hemolytic assays for depletion of serum complement activity and induction of bystander lysis can be employed for screening.

### Complement Depletion:

To measure the anticomplementary (complement consumption) activity of modified human C3 proteins, a small volume of human serum is incubated with CVF or modified human C3 proteins for three hours or shorter periods of time at 37° C. at a protein concentrations ranging from 1 to 3200 ng/μl (such as 5 μg/ml), to allow the proteins to deplete complement. The remaining complement hemolytic activity is subsequently measured using sensitized sheep erythrocytes using methods known to one of skill in the art including that of Cochrane et al., 1970 (Cochrane et al., 1970, J. Immunol. 117:630-4).

For example, the assay may be performed in two steps. In the first step, the protein of interest is diluted to the desired concentrations in buffer, usually by serial dilution (typically from less than a nanogram/microliter up to approximately 320 ng/microliter or 3.2 μg in the 10 microliters used in the assay). Then, a 1 μl or 10 μl aliquot of the diluted protein is mixed with undiluted serum. The mixture is incubated at 37° C. for 3 hours, which allows the protein to activate complement by forming a C3 convertase. The convertases formed are then able to activate C3 in the serum. Then, to measure the amount of complement activity left, the serum is diluted and mixed with antibody-sensitized sheep erythrocytes, which are easily lysed by complement when it is present in serum. This reaction is allowed to proceed for 30 minutes, and is stopped by diluting the mixture in cold buffer. The cells are centrifuged, and the lysed cells are quantified by measuring the hemoglobin released.

Complement depletion of modified human complement C3 proteins is additionally measured using surface plasmon resonance (SPR).

### Bystander Lysis Assay.

The bystander lysis assay is performed by incubating 20 μl of normal guinea pig serum at 37° C. with 20 μl of CVF or modified human C3 proteins at a concentrations between 1 and 200 μg/ml (such as 5 μg/ml) and 20 μl guinea pig erythrocytes (5×100/ml). The CVF or modified human C3 proteins participate in fluid-phase activation of C5, which leads to lysis of the erythrocytes. Thus, presence of hemoglobin in the supernatant is indicative of C5 activation. The reaction is incubated at 37° C. for 30 minutes, and is stopped by the addition of 1 ml of cold buffer. After centrifugation, the released hemoglobin is measured spectrophotometrically. (Vogel, C. W., and Müller-Eberhard, H. J. (1984) J. Immunol. Methods 73(1), 203-220, herein incorporated by reference in its entirety).

### C3 Convertase Formation/Factor B Activation

To detect cleavage of Factor B into Ba and Bb, a modified human C3 protein (at 1 μM) is incubated for up to twenty four hours in the presence of a three-fold molar excess of Factor B and 0.5 μM of Factor D in the presence of MgCl₂ at 37° C. The reaction mixtures are analyzed by electrophoresis on 7.5% (w/v) SDS polyacrylamide gels under non-reducing conditions to monitor the disappearance of Factor B and the appearance of the cleavage products Ba and Bb. If necessary, a subsequent western blot can be performed to detect the Ba and Bb cleavage fragments. Controls can include native CVF, pro-CVF, cobra C3, iC3, human C3, iC3, C3b, and EDTA (Vogel and Müller-Eberhard, 1982, J. Biol. Chem. 257:8292-9).

C3 convertase formation of modified human complement C3 proteins is additionally measured using surface plasmon resonance. The proteins are bound to a BIACORE CHIP™, and then contacted with complement factors. The amount of each of the complement factors bound to the chip (and thus bound to the modified human C3 proteins) is determined by surface plasmon resonance.

Factor B cleavage of modified human complement C3 proteins is additionally measured using surface plasmon resonance. A factor B cleavage graph indicates a time course of the ability of the modified human complement C3b proteins to mediate the cleavage of factor B in the presence of magnesium ion and factor D. This can be used to measure the ability of the protein to form a C3/C5 convertase.

### C3 Convertase Activity Assay

To examine C3 cleaving activity, a C3 convertase is pre-formed as described herein, in reference to “C3 convertase formation/Factor B activation,” using the modified human C3 proteins and human Factor B and Factor D. This assay measures the activity of C3/C5 convertases containing modified human C3 proteins to activate human C3, by cleaving off the C3a peptide. The convertase formation is stopped by the addition of EDTA, and purified human C3 is added. The reaction mixture is incubated at 37° C. for one hour or for any other appropriate period of time. Aliquots are taken and immediately transferred into an ice water bath to stop further C3 activation. C3 cleavage is monitored by the disappearance of the C3 α-chain and appearance of the C3 α′-chain by running the reaction products on a 7.5% (w/v) SDS polyacrylamide gel under reducing conditions. If necessary, a subsequent western blot using anti-C3 antiserum is performed. Controls include native CVF, pro-CVF, and human and cobra iC3 or C3b (Vogel and Müller-Eberhard, 1982, J. Biol. Chem. 257:8292-9).

### C5 Conversion Assay

The assay for C5 conversion activity is done essentially as described by Petrella et al., (1987) J. Immunol. Methods 104(1-2), 159-172. In this assay, C5 convertase is formed as described above, using a total of 3 μg protein. After convertase formation, the reaction is stopped by the addition of EDTA to a final concentration of 5 mM. Then, 5 μl of this reaction is added to a 25 μl reaction containing 7 μg C5 in PBS. The reaction is incubated at 37° C. for 24 hours, and the reaction is stopped by the addition of 7 μl Laemmli gel loading buffer, followed by boiling for 5 minutes. The reaction products are separated by reducing SDS-PAGE, and the gel is stained with Coomassie Blue dye, and the relative amounts of the C5 α-chain and C5 α′-chain quantified as described above.

The assay for factor degradation of the proteins by factors H and I is performed essentially according to the method of Oran and Isenman ((1999) J. Biol. Chem. 274 (8), 5120-5130). In this method, 12 μg of each protein is incubated with 4.3 μg factor H and 0.3 μg factor I at 37° C. in a total volume of 60 μl. At the indicated times, 10 μl aliquots are withdrawn, and reactions are stopped by the addition of 5 μl 5× Laemli gel loading buffer. Reaction products are separated by SDS-PAGE on a 4-20% gradient gel under reducing conditions.

### Assay for Convertase Stability and Intrinsic Half-Life

Bimolecular convertases are pre-formed using the modified human C3 proteins and purified human Factor B and Factor D as described above. After addition of EDTA, the mixture is incubated at 37° C. and aliquots are removed over a period of 24 hours or shorter periods of time if appropriate, and each aliquot is immediately placed in an ice water bath. Subsequently, the C3 convertase activity is determined by the C3 cleaving assay described above. From the reduction of the C3 cleaving activity over time, the half-life of the spontaneous decay-dissociation of the various convertases is calculated. If insufficient quantities of modified human C3 proteins for this assay are available, the enzymatic activity is determined using the fluorogenic tripeptide t-butyloxy-carbonyl-leucyl-glycyl-arginyl-aminomethylcoumarin. (Caporale, L. H., Gabaer, S. S., Kell, W., and Gotze, O. 1981 J. Immunol. 126(5), 1963-1965, herein incorporated by reference in its entirety).

Surface plasmon resonance (SPR) may also be used to determine the convertase stability and intrinsic half-life formed by the modified C3 proteins. A modified C3 protein of interest is attached to a chip, and then exposing the chip to factor B and factor D in the presence of Magnesium. Then, factor B and D are removed by flowing buffer over the chip. Surface plasmon resonance measures the loss of Bb bound to the modified C3 protein attached to the chip over time.

Alternatively, EDTA may be added to stop the formation of convertase; and then, at various times, aliquots are removed and the activity of the convertase measured by performing C3 cleavage reaction. It is also possible to obtain approximate stabilities by observing the rate of factor B cleavage. For example, if factor B is present in a three-times molar excess over the modified C3 protein, the rate of factor B cleavage is dependent on the dissociation of already formed convertase. For rapidly dissociating convertase, observing the C3 cleavage reaction at low convertase concentrations allows one to estimate the half-life of the convertase.

### Assay for Factor H Binding.

Factor H binding to modified human C3 proteins is determined using an ELISA assay. Modified human C3 proteins are adsorbed onto microtiter plates. After blocking with ovalbumin and BSA, purified human Factor H at 10 μg/ml is added and incubated for 30 minutes at room temperature. After washing, bound Factor H is detected with anti-Factor-H antibody followed with an appropriate phosphatase-linked secondary antibody. If factor H can bind to the protein, an appropriate color change is observed. Controls can include native CVF, pro-CVF, cobra and human C3, as well as cobra and human iC3 (Alsenz et al., 1992, Dev. Comp. Immunol. 16:63-76).

### Assay for Factor I Cleavage

Modified human C3 proteins are incubated with purified human Factor H and Factor I at 37° C. for several hours. The reactions are analyzed by subsequent 7% (w/v) SDS polyacrylamide gel electrophoresis under reducing conditions. Factor I activity is determined by the reduction in the strength of the 105 kDa α′-chain band, and appearance of bands with a molecular weight of 37 and 40 kDa. If necessary, a subsequent western blot is performed using anti-CVF and/or anti-C3 antibodies. Alternatively, modified human C3 proteins are labeled with ¹²⁵I using the iodogen method (Fraker and Speck, 1978). Cleavage products are detected after SDS polyacrylamide gel electrophoresis by autoradiography (Lambris et al., 1996, J. Immunol. 156:4821-32).

### Assays for Immunogenicity

Various methods can be used to analyze immunogenicity, including but not limited to, skin tests, testing the modified human C3 protein in transgenic animals which have been genetically engineered to have human immune systems, in vitro methods, including RIA tests using serum generated in such transgenic animals, Radioimmunoprecipitation assays, ELISA assays, Electrochemiluminescence, and Surface Plasmon Resonance. In addition, mouse, rate or guinea pig analogs of some proteins are constructed, using either mouse, rate or guinea pig C3 and CVF sequences. These are injected into the appropriate animal, and serum is collected and analyzed for the production of antibodies against the modified human C3 proteins.

### Method of Measuring Plasma Half-Life

There are many factors that can affect the plasma half-life of the modified human C3 protein, including but not limited to specific antibodies produced by the immune system, proteases circulating within the serum, non-specific immune responses, and specific regulatory factors such as Factors H and I. In order for the modified human C3 proteins to be able to activate and subsequently deplete complement, preferred C3 proteins will persist within human plasma for at least a minimum amount of time. Thus, it is of interest to identify the plasma half-life of the modified human C3 proteins to determine how useful they will be for treatment of certain diseases. This method measures the stability of the modified human C3 protein in plasma in three ways. However it is to be understood that one or all of the methods can be used as well as any other methods known to one of skill in the art.

The first method measures the stability in serum in vitro. Human serum is isolated and separated from the whole blood of a patient. Aliquots of different concentrations of the modified human C3 proteins are added to the serum and allowed to incubate. Aliquots of the serum are removed at various time intervals and the amount of modified human C3 that persists is identified in an ELISA assay using a monoclonal antibody which is specific to C3.

A second method allows for the identification of stability in serum in a humanized animal. The modified human C3 protein is administered to the animal and blood samples are taken over time. The amount of modified human C3 protein is identified in an ELISA assay using specific antibodies to the protein.

A third method allows for the identification of stability in a human patient. The modified human C3 is administered to the patient and blood samples are removed over time. The amount of modified human C3 protein is identified using an ELISA assay. This will give a clear indication of how long the modified human C3 protein circulates within the plasma of a patient.

In some embodiments, the antibody that is used need not be specific for the modified human C3. For example, antibodies that recognize normal C3 can be tested and used to identify the modified human C3 in an ELISA procedure.

### Example 4

### Expression of Modified Human C3 Proteins and Activity Measurements of the Modified Human Complement C3 Proteins

The modified human C3 proteins were produced in the Drosophila S2 cell system. Briefly, the plasmids (described in Example 1) were transfected into Drosophila S2 cells using the calcium phosphate method of Chen and Okayama (Chen, C., and Okayama, H. (1987) Mol. Cell. Biol. 7(8), 2745-2752). S2 cells were transfected with a mixture of expression plasmid and pCoBlast, using a ratio of 19:1 (w:w). Following transfection, cells containing both plasmids were selected using blasticidin (25 μg/ml). For expression, 1-liter cultures of transfected cells were grown in serum-free medium (Hi-Five plus L-glutamine), in the absence of blasticidin. When the cells reach a density of 5×10⁶ cells/ml, production of the recombinant proteins was induced by the addition of 10 mM CdCl₂ for 5 days. Proteins were purified by a combination of DEAE, BioGel, and ANX chromatography. The modified human C3 proteins were then tested for activity.

### Assay for Factor B Cleavage

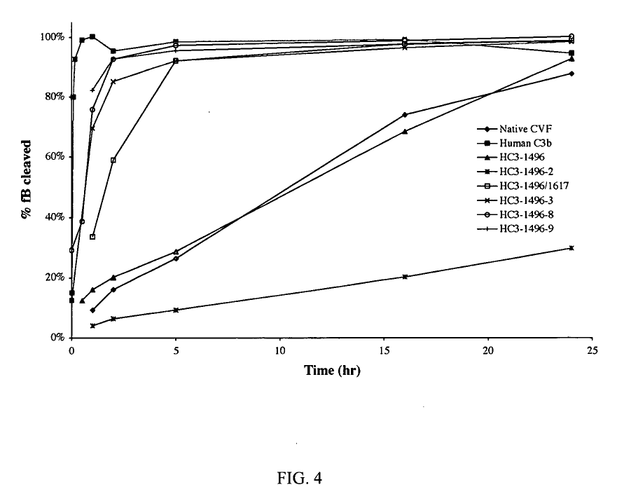

The modified human C3 proteins including HC3-1496, HC3-1496-2, HC3-1496-3, HC3-1496/1617, HC3-1496-8, and HC3-1496-9 or control (native CVF or Human C3b) at a concentration of 1 μM were incubated at 37° C. with a three-fold molar excess of factor B and factor D (0.08 μM) in PBS plus 10 mM MgCl₂. At the times indicated, 10 μl aliquots were removed and the reaction stopped by heating in the presence of Laemmli buffer. Reaction products were separated by non-reducing SDS-PAGE on 7.5% gels. Gels were stained with Comassie Blue, scanned, and bands quantified using Quality One software from Bio-Rad. FIG. 4 provides the results of the Factor B cleavage assay.

### Assay for C3 Conversion Activity

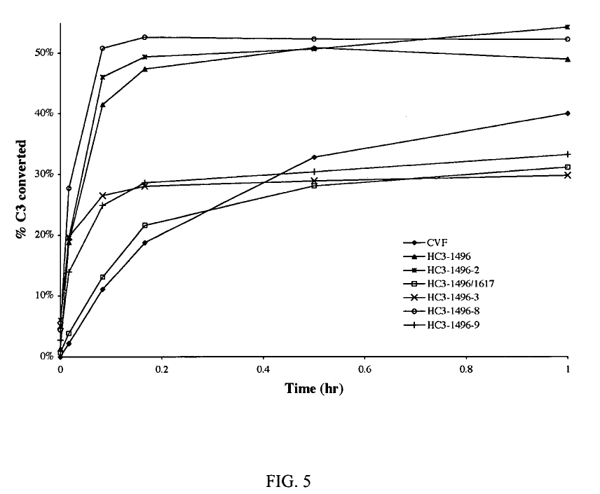

This assay measured the activity of C3/C5 convertases containing modified human C3 proteins to activate human C3, by cleaving off the C3a peptide. To examine C3 cleaving activity, a C3 convertase was pre-formed using the modified human C3 proteins and human Factor B and Factor D. The convertase was pre-formed as described above for the Assay for Factor B Cleavage for modified human C3 proteins including HC3-1496, HC3-1496-2, HC3-1496-3, HC3-1496/1617, HC3-1496-8, and HC3-1496-9 or a control (native CVF) at a concentration of 1 μM. Convertase formation was stopped by the addition of EDTA to a final concentration of 10 mM. The length of the reaction to form the convertases was calculated so that all preparations contained the same amount of convertase. To analyze the ability of the convertase to cleave human C3, preformed convertases were added to 5 μg human C3, and the reaction incubated in PBS+10 mM EDTA at 37° C. At indicated times, 10 μl aliquots were removed, and the reaction stopped by boiling the reaction in Laemmli buffer containing 5% β-mercaptoethanol as a reducing agent. Reaction products were separated by SDS-PAGE on 6% gels. Gels were stained with Comassie Blue, scanned, and amount of C3 cleavage was determined by quantifying bands using Quality One software from Bio-Rad. FIG. 5 provides the results of the C3 conversion activity assay.

### In Vitro Complement Depletion Assay

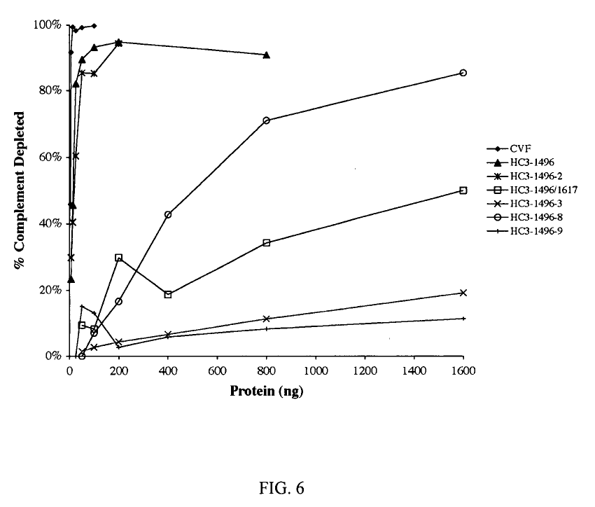

The modified human C3 proteins including HC3-1496, HC3-1496-2, HC3-1496-3, HC3-1496/1617, HC3-1496-8, and HC3-1496-9 or a control (native CVF) were serially diluted into TBS-G (TBS buffer containing 0.5% gelatin). Serum complement was depleted by the addition of 1 μl of protein (at protein amounts of between 1 and 1600 ng as shown in FIG. 6) to 10 μl of NHS, and the reactions were incubated for 3 hours at 37° C. Reactions were stopped by addition of 49 μl of cold TBS-G. Remaining serum complement was measured by determining the ability of 10 μl of the diluted serum to lyse antibody-sensitive sheep erythrocytes (30 μl of cells at 5×10⁸ cells/ml) in a 30 minute reaction at 37° C. in a 150 μl reaction. Reactions were stopped by the addition of 1 ml cold TBS-G and the amount of lysis was determined by measuring the hemoglobin release spectorphotometrically at 412 nm. FIG. 6 provides the results of the in vitro complement depletion assay.

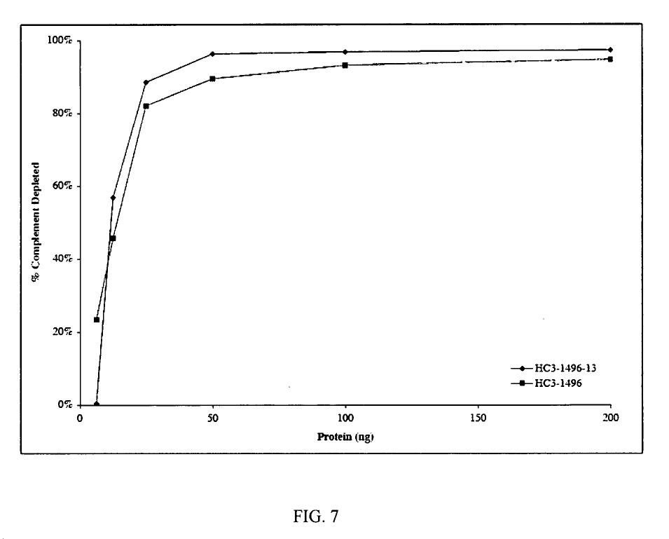

In another experiment, the modified human C3 proteins including HC3-1496-13 or a control (native CVF) were serially diluted into TBS-G (TBS buffer containing 0.5% gelatin). Serum complement was depleted by the addition of 1 μl of protein (at protein amounts of between 1 and 1600 ng as shown in FIG. 7) to 10 μl of NHS, and the reactions were incubated for 3 hours at 37° C. Reactions were stopped by addition of 49 μl of cold TBS-G. Remaining serum complement was measured by determining the ability of 10 μl of the diluted serum to lyse antibody-sensitive sheep erythrocytes (30 μl of cells at 5×10⁸ cells/ml) in a 30 minute reaction at 37° C. in a 150 μl reaction. Reactions were stopped by the addition of 1 ml cold TBS-G and the amount of lysis was determined by measuring the hemoglobin release spectrophotometrically at 412 nm. FIG. 7 provides the results of the in vitro complement depletion assay using HC3-1496-13 or CVF.

In Vivo Complement Depletion with CVF and HC3-1496

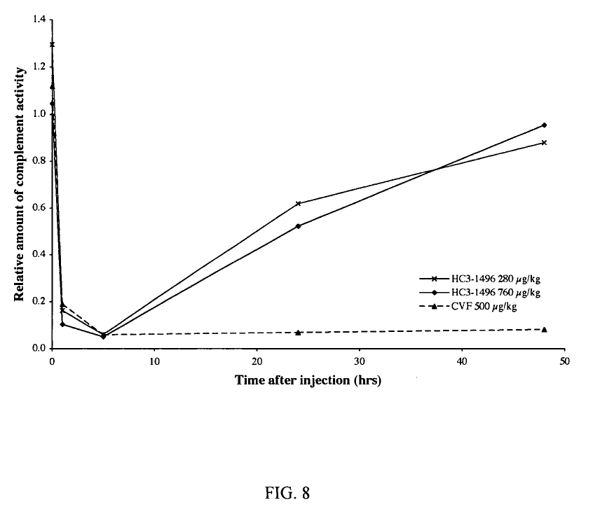

Male Sprague-Dawley rats were injected i.p. with either 500 μg/kg of CVF (n=1), 760 μg/kg of HC3-1496 (n=3), or 280 μg/kg of HC3-1496 (n=3). At various time points as shown in FIG. 6, blood was obtained by nicking the tail vein. The blood was allowed to coagulate, and the clots removed by centrifugation. Serum samples were assayed for complement activity by measuring the ability of the serum to induce lysis of antibody-sensitive sheep erythrocytes as described in the in vitro complement depletion assay. FIG. 8 provides the results of the in vivo complement depletion with CVF and HC3-1496.

### Results

As shown in FIG. 6, HC3-1496-2 possessed complement depleting activity which was very similar to HC3-1496, but formed a much more stable convertase (see FIG. 4 for rate of convertase formation in Assay for Factor B Cleavage).

As shown in FIG. 6, HC3-1496-8 was able to deplete complement, though not as well as HC3-1496, and the convertase containing this protein was much less stable than the HC3-1496-containing enzyme as shown in FIG. 4. The HC3-1496-8-containing convertase, as shown in FIG. 5, however, was even more active in cleaving C3 than was the HC3-1496-containing enzyme. By substituting parts of the region between 1504 and 1550, this hybrid showed that it should be possible to prepare a protein that forms a more stable, more active convertase than does HC3-1496.

As shown in FIG. 7, HC3-1496-13 depleted complement as well as HC3-1496 and showed that these residues do not cause a major effect on the stability or activity of the C3 convertase.

HC3-1496-9, as shown in FIG. 4, further demonstrated the importance of the amino acids at positions 1571, 1576, 1577, and 1578, in that substituting the amino acid residues found in human C3 into these positions results in a very unstable convertase.

As shown in FIG. 4, HC3-1496/1617 demonstrated that the C-terminal region from 1617 to 1663 was important for the formation of a stable convertase.

HC3-1496-3 showed that the substitution of one amino acid residue (residue 1633) can have a profound effect on convertase stability. See FIG. 4. HC3-1496-3 contained one additional residue found in CVF than HC3-1496/1617, yet formed a much less stable convertase.

The various methods and techniques described above provide a number of ways to carry out the invention. Of course, it is to be understood that not necessarily all objectives or advantages described may be achieved in accordance with any particular embodiment described herein. Thus, for example, those skilled in the art will recognize that the methods may be performed in a manner that achieves or optimizes one advantage or group of advantages as taught herein without necessarily achieving other objectives or advantages as may be taught or suggested herein.

Furthermore, the skilled artisan will recognize the interchangeability of various features from different embodiments. Similarly, the various features and steps discussed above, as well as other known equivalents for each such feature or step, can be combined and/or exchanged by one of ordinary skill in this art to perform methods in accordance with principles described herein. Each patent, journal reference, and the like, cited herein is hereby incorporated by reference in its entirety.

Although the invention has been disclosed in the context of certain embodiments and examples, it is understood by those skilled in the art that the invention extends beyond the specifically disclosed embodiments to other alternative embodiments and/or uses and obvious modifications and equivalents thereof. Accordingly, the invention is not intended to be limited by the specific disclosures of preferred embodiments herein.

1. A modified human C3 protein, comprising a human C3 protein, wherein amino acid residues in the human C3 protein corresponding to amino acid residues about 1496 to about 1663 of SEQ ID NO:1 are substituted with a corresponding portion of a Cobra Venom Factor (CVF) protein, wherein the CVF portion of the modified human C3 protein comprises a further substitution of one or more amino acid residues in one or more regions selected from the group consisting of amino acid residues about 1499 to about 1501, amino acid residues about 1507 to about 1510, amino acid residues about 1519 to about 1550, and amino acid residues about 1596 to about 1617.

2. The modified human C3 protein of claim 1, wherein the further substitution of one or more amino acid residues is in the region of amino acid residues about 1499 to about 1501.

3. The modified human C3 protein of claim 2, wherein the further substitution of one or more amino acid residues is a substitution with one or more corresponding amino acids of SEQ ID NO:1 or conservative substitutions thereof.

4. The modified human C3 protein of claim 3, wherein the further substitution of one or more amino acid residues is a substitution with one or more corresponding amino acids of SEQ ID NO:1.

5. The modified human C3 protein of claim 2, wherein the further substitution of one or more amino acid residues is T1499X and L1501Z, wherein X is amino acid residue D or a conservative substitution thereof and Z is amino acid residue K or a conservative substitution thereof.

6. The modified human C3 protein of claim 5, wherein the further substitution of one or more amino acid residues is T1499D and L1501K.

7. The modified human C3 protein of claim 1, wherein the further substitution of one or more amino acid residues is in the region of amino acid residues about 1519 to about 1550.

8. The modified human C3 protein of claim 7, wherein the further substitution of one or more amino acid residues is a substitution with one or more corresponding amino acids of SEQ ID NO:1 or conservative substitutions thereof.

9. The modified human C3 protein of claim 8, wherein the further substitution of one or more amino acid residues is a substitution with one or more corresponding amino acids of SEQ ID NO:1.

10. The modified human C3 protein of claim 7, wherein the further substitution of one or more amino acid residues is a substitution with corresponding amino acids about 1519 to about 1550 of SEQ ID NO:1 or conservative substitutions thereof.

11. The modified human C3 protein of claim 10, wherein the further substitution of one or more amino acid residues is a substitution with corresponding amino acids about 1519 to about 1550 of SEQ ID NO:1.

12. The modified human C3 protein of claim 1, wherein the further substitution of one or more amino acid residues is in the region of amino acid residues about 1598 to about 1600.

13. The modified human C3 protein of claim 12, wherein the further substitution of one or more amino acid residues is V1598X, N1599Z1, and D1600Z2, wherein X is amino acid residue L or a conservative substitution thereof, Z1 is amino acid residue D or a conservative substitution thereof, and Z2 is amino acid residue N or a conservative substitution thereof.

14. The modified human C3 protein of claim 13, wherein the further substitution of one or more amino acid residues is V1598L, N1599D, and D1600N.

15. The modified human C3 protein of claim 1, wherein the further substitution of one or more amino acid residues is in the regions of amino acid residues about 1499 to about 1501 and amino acid residues about 1519 to about 1550.

16. A composition comprising the modified human C3 protein of claim 1.

17. (canceled)

18. A polynucleotide comprising a nucleic acid sequence encoding the modified human C3 protein of claim 1.

19. A vector comprising the polynucleotide of claim 18.

20. A cell comprising the vector of claim 19.

21. A method for producing the modified human C3 protein of claim 1 comprising culturing a host cell comprising a polynucleotide comprising a nucleic acid sequence encoding the modified human C3 protein under a condition that the modified human C3 protein is expressed; and purifying the expressed modified human C3 protein.

22. (canceled)

23. A method for depleting complement in an individual comprising administering to the individual a modified human C3 protein of claim 1 in an amount effective for the depletion of complement.

24. (canceled)
